(19) United States
(12) Patent Application Publication
(10) Pub. No.: US 2019/0367904 A1
(43) Pub. Date: Dec. 5, 2019

(54) AUTOMATED METHOD FOR RELEASE OF NUCLEIC ACIDS FROM MICROBIAL SAMPLES

(71) Applicant: ZYMO RESEARCH CORPORATION, Irvine, CA (US)

(72) Inventors: Ryan KEMP, Irvine, CA (US); Xi-Yu JIA, Newport Beach, CA (US); Yakov KAPLAN, San Diego, CA (US); Brandon POLLACK, Northridge, CA (US)

(21) Appl. No.: 16/348,115

(22) PCT Filed: Nov. 7, 2017
(86) PCT No.: PCT/US2017/060345

§ 371 Date: May 7, 2019

Related U.S. Application Data
(60) Provisional application No. 62/418,762, filed on Nov. 7, 2016.

(51) Int. Cl. C12N 15/10; C12N 1/06; C12N 13/00; C12M 1/00

(52) U.S. Cl. CPC C12N 15/1013; C12M 47/06; C12N 13/00; C12N 1/066

(57) ABSTRACT

Methods and devices are provided for reduced biased isolation of genetic materials from mixed microbial samples are provided.

This application is a national phase application under 35 U.S.C. § 371 of International Application No. PCT/US2017/060345, filed Nov. 7, 2017, which claims the benefit of U.S. Provisional Patent Application No. 62/418,762, filed Nov. 7, 2016, the entirety of each of which are incorporated herein by reference.

### BACKGROUND OF THE INVENTION

### 1. Field of the Invention

The present invention relates generally to the field of molecular biology. More particularly, it concerns nucleic acid isolation, particularly the isolation of DNA.

### 2. Description of Related Art

In the case of genomic DNA, modem molecule biological techniques require substantially purified DNA samples and, in some cases it is highly desirable to purify genomic material having a limited amount of retained RNA and/or plasmid DNA. Likewise, in the purification of plasmid DNA from bacterial lysates, plasmid purity is important for downstream recombinant DNA manipulations. The sensitive reactions commonly employed in molecular biology experiments of reverse transcription, transcription, DNA and RNA sequencing, polymerase chain reaction (PCR), restriction digests, ligation reactions, end modifications, among other similar base modification procedures require the DNA, or other nucleic acid molecules, be essentially free from contaminants. It is also desirable to isolate the nucleic acid in significant quantities to ensure a reliable source of material with which to proceed to additional experiments. In many instances there is a need to move a desired DNA, or fragment thereof, through several manipulations to reach the desired endpoint. Cloning procedures, for example, are often complex and involve numerous steps; therefore, methods that reliably isolate pure DNA, and other nucleic acids, in significant quantities are desired.

Conventional procedures for isolating plasmid DNA, for example, include harvesting the bacterial cells and obtaining the plasmid DNA, or other target nucleic acid, in a pure form via lysis, free from undesirable contaminating medium and cellular constituents. This is typically called a cleared bacterial or cellular lysate. The cell lysis may be performed in a variety of ways including mechanical sonication or blending, enzymatic digestion and also the traditional chemical means of alkaline lysis. The alkaline lysis based protocols remain the basis for many plasmid purification methods, though other procedures, such as the boiling lysis, triton lysis, and polyethylene glycol protocols, are also used (Bimboim and Dolly, 1979; Bimboim, 1983; Holmes and Quigley, 1981; Clewell and Helinski, 1970; Lis and Schleif, 1975).

Genomic DNA isolated from blood, tissue or cultured cells has several applications, which include PCR, sequencing, genotyping, hybridization and Southern Blotting. Most protocols for the preparation of bacterial genomic DNA consist of lysis, followed by incubation with a nonspecific protease and a series of extractions prior to precipitation of the nucleic acids. A common method comprises a first step of enzymatic lysis of the microbial cells, followed by extraction of the DNA, binding the extracted DNA to magnetic beads, immobilizing the beads with a magnet, washing away the non-DNA components in the sample, and eluting the now purified DNA from the beads into a PCR compatible buffer solution.

Approaches that coupled alkaline lysis to cesium chloride gradient centrifugation and organic extraction with toxic and caustic phenol/chloroform and alcohols have largely been replaced by a variety of systems that use rapid and efficient chromatographic methods. The observation that DNA bound preferentially to ground glass or glass fiber disks in the presence of high concentrations of sodium iodide or sodium perchlorate allowed the development of new purification methodologies (Marko et al., 1981; Vogelstein et al., 1979). The use of the chaotropic salt solutions, such as guanidinium, iodide, perchlortate, and trichloroacetate, coupled to forms of silica-based or other chromatographic techniques, has resulted in a methodology for plasmid as well as general nucleic acid purification.

Advancements in NGS as well as increased funding have enabled, large-scale, multi-lab research of microbial communities. However, early quality control studies on microbiomics research suggest that, while the technology and funding are readily available, there are no standard reference materials or controls. The field is littered with potential sources for error and bias. The combination of variation in measurements between labs and lack of standard reference materials have led to growing concern within the scientific community about the reproducibility of research (Sinha et al., 2015).

In the nucleic acid extraction phase, inferior forms of lysis can fail to extract DNA uniformly from a diverse sample of microbes (Kennedy et al., 2014). Chemical, enzymatic, and many lysis matrices lead to inferior lysis and an unrealistic representation of the microbial community (Zoetendal et al., 2001). This is especially the case for tough-to-lyse microbes, leading to their underrepresentation and the overrepresentation of easy-to-lyse microbes in any given sample. Mechanical lysis currently offers the most accurate representation of a microbial community (Ariefdjohan et al., 2010). However, not all mechanical lysis methodologies perform equally. The type of bead (matrices) used to homogenize the sample and the type of homogenization device are major contributors to bias. Many mechanical lysis protocols using extremely high speed disruptors, that are not fully optimized, fail to uniformly lyse a wide array of different organisms ranging in size and hardiness, including bacteria, yeast, and spores (Kennedy et al., 2014).

Automated nucleic acid extraction systems currently available include bead movers, liquid handlers, plate movers or centrifuges, microfluidics, or other pressurized liquid transfer mechanisms. Systems typically also include functions such as shaking, temperature control, and PCR protocols. For example, systems include the InnuPure® Automated Nucleic Acid Extraction System, VERSA 10 NAP Automated Nucleic Acid Purification Workstation, and the Chemagic™ 360 Nucleic Acid Extractor.

However, automated systems for nucleic acid extraction have not integrated tools to homogenize and lyse tough-to-lyse organisms with the exception of highly costly robotic platforms that use a robotic arm (e.g., Thermo Scientific's VALet™ robotic arm) to link high speed disruptors (e.g., Spex SamplePrep 2025 Geno/Grinder) to a purification platform (e.g. KingFisher™ Purification Systems). The cost of said methodology becomes prohibitive for many groups in need of automation. A Nature Microbiology consensus statement published in January of 2016 identified “standardized protocols and high throughput tools” as commonly listed needs by the community meaning the previously described automation did not resolve the problems of the Microbiomics community (Stulberg et al., 2016).

It has been stated that microbiome research to date has been difficult to reproduce and that variation at each step is enormous meaning the need for standardized highly reproducible procedures is currently an unmet need (Sinha et al., 2015). Automation is part of the solution to this problem, because it removes the user from the equation, however existing automated solutions face the large problem of bias associated with inefficient lysis which can lead to misrepresentative data and overall poor results as wells as signal dropouts (e.g., false negatives). Despite these improvements and the development of numerous nucleic acid purification systems there remains a need to develop improved systems to satisfy demands for easier, faster lower cost system with increased yield and reliability, and unbiased purification (extraction of nucleic acids that is reflective of the community actual population) of nucleic acid materials from mixed microbial samples.

### SUMMARY OF THE INVENTION

In a first embodiment, the present disclosure provides an automated method for reduced-biased nucleic acid isolation from a plurality of samples comprising disposing the plurality of samples into an array of sample containers, and applying a mechanical force to the sample containers, wherein each sample container comprises the sample and a bead or a plurality of beads for disrupting microbial cells and viruses, thereby providing a unbiased release of microbial nucleic acids.

In some aspects, the plurality of beads are loaded in the sample container at 40-60% by volume, such as about 40%, 45%, 50%, 55%, or 60% bead loading. In some aspects, the plurality of beads comprised of beads of different materials, sizes, or different shapes or the combination thereof. In certain aspects, the beads are substantially spherical and comprise an average diameter of between 0.01 and 1.0 mm. In particular aspects, the beads of different sizes comprise beads that are between 0.25 and 0.75 mm and beads that are between 0.05 and 0.25 mm in diameter. In some aspects, the beads of different sizes comprise a mixture of beads of 0.1 mm and 0.5 mm diameter beads, such as at a ratio of 1:1, 1:2, 1:3, 1:4, 1:5, 1:6 or 2:1, 3:1, 4:1, 5:1, 6:1 by volume. In particular aspects, the bead is substantially spherical. In some aspects, the bead is composed of a substantially non-reactive material. In one particular aspect, the bead is composed of a ceramic.

In some aspects, the sample containers are in a 24-well, 48-well, or 96-well format. In particular aspects, the sample containers are in a 24-well format.

In certain aspects, the plurality of samples are comprised of but not limited to viruses, bacterial cells, fungal cells, algal cells, plant cells, animal cells, archaeal cells, protozoans or a mixture thereof. In some aspects, the plurality of samples each comprise at least two, three or four different types of biological agents selected from the group consisting of viruses, bacteria cells, fungal cells, spores, plant cells, animal cells and archaeal cells. In particular aspects, the plurality of samples are comprised of environmental samples including but not limited to water, biofilms, soil, air or host derived samples including but not limited to body fluids, saliva, urine fecal, root, leaf or bark samples and any surface or liquid that can be swabbed.

In particular aspects, applying a mechanical force comprises subjecting the sample container to oscillation, such as lateral oscillation, horizontal oscillation, vertical oscillation, orbital or a mixture thereof. In one specific aspect, applying a mechanical force comprises moving the sample container with a shaker.

In some aspects, the sample container further comprises a bashing magnet. In particular aspects, the bashing magnet is rectangular or cylindrical. The bashing magnet may be a rectangular bar. In some aspects, the bashing magnet and edge of the sample container comprise a gap of 0.7 to 1.2 mm, such as 0.9 to 1.1 mm, particularly about 1.0 Mm. In certain aspects, subjecting the sample container to oscillation comprises using a drive magnet to move the bashing magnet. In some aspects, the drive magnet produces a spinning magnetic field. In particular aspects, magnet polarity is normal to the spinning magnetic field. In some aspects, center positions of the drive plate and sample containers are aligned with spinning axes of the drive magnet. In specific aspects, the drive magnet to bashing magnet pulling force ratio is from about 5:1 to 20:1 or more preferably, 8:1 to 10:1. In some aspects, the oscillation, such as lateral oscillation, has an offset of 10-50 mm or more preferably 15-25 mm. In some aspects, subjecting the sample to oscillation comprising using an oscillating drive plate.

In additional aspects, the sample container further comprises a lysis buffer. In some aspects, the lysis buffer comprises a reagent that can facilitate binding including but not limited to chaotropic salts, kosmotropic salts, alcohol, PEG, and cationic detergents.

In some aspects, the unbiased release of microbial nucleic acids is at least 80%, such as at least 85%, 90%, 95%, 96%, 97%, 98%, 99%, or 100% of nucleic acids in the sample.

In some aspects, the method further comprises purification of nucleic acids from tough to lyse samples especially complex mixed community samples and microbial samples. In certain aspects, purification comprises capturing the microbial nucleic acids on a surface, washing the captured microbial nucleic acids with a wash buffer, and eluting the microbial nucleic acids from the surface. In some aspects, capturing comprises loading the nucleic acids onto a glass fiber matrix on a vacuum/spin column or plate. In other aspects, capturing comprises binding the nucleic acids to magnetic microparticles wherein a preferred embodiment is silica microparticles.

A further embodiment provides a kit comprising a plurality of high density beads; a device for applying an automated mechanical force to sample container; and a control sample. In some aspects, the control sample comprises a mixed microbial population having a known proportion of each microbial component. In certain aspects, the device for applying an automated mechanical force to sample container comprises a shaker. In some aspects, further comprising instructions, a lysis buffer, a binding buffer, nucleic acid binding beads and/or a stabilization buffer. In some aspects, the mechanical force is set by the controller.

In yet another embodiment, there is provided a device for the purification of nucleic acids comprising an automated system comprising a plurality of sample containers, each container comprising cell lysis beads and a system for providing mechanical force to the containers, wherein the device provides lysis of microbial samples with a reduced bias. In some aspects, the system for providing mechanical force to the containers comprises bashing magnets in each sample container and a drive magnet providing a spinning magnetic field on said bashing magnets. In particular aspects, the bashing magnets are rectangular or cylindrical, such as a bar or rod. In some aspects, the distance between the bashing magnets and edge of the sample container is 0.7 to 1.2 mm, such as about 0.9 to 1.1 mm, particularly about 1.0 mm. In some aspects, the spinning magnetic field results in lateral oscillation and/or orbital oscillation. In particular aspects, the lateral oscillation and/or orbital oscillation have an offset 15-25 mm.

As used herein, an “unbiased”, “limited bias”, or “reduced-biased” microbial nucleic acid isolation refers to a method that isolates genetic material from a mixed microbial sample such that the resulting amount of genetic material from each of the microbial populations within a community is extracted in an abundance relative to each other that accurately reflects the prevalence of each of the given microbial cells in the sample. For example, a reduced-biased method can be defined as able to isolate genetic material that allows for relative quantitation of a given microbial cell component in the sample with 30%, 20%, 10% or 5% of the actual prevalence of the constituent in the sample. This can be measured through the use of mock microbial community of known composition comprising organisms representing a range of recalcitrance to lysis, such as different gram-stains, cell wall structures, cell wall composition, and organism sizes. The purified nucleic acids should reasonably accurately reflect the relative abundances the organisms within the standard. Examples of analytical methods used to evaluate the efficiency of a system to accurately purify DNA from a mixed population is 16s rRNA Gene Sequencing and or Shotgun metagenomic sequencing.

The term “efficient lysis” refers to efficiently breaking open cellular membranes. Lysis can be chemical lysis (e.g., detergents, chaotropic salts, phenol and other organic solvents), enzymatic lysis (e.g., proteases to denature and degrade proteins that interact with nucleic acids), and/or mechanical lysis (e.g., physical grinding or bead beating). Lysis of microbial samples can be performed using enzymatic methods (e.g., lysozyme, lysostaphin, and mutanolysin) that specifically break down peptidoglycan cell walls. Mechanical lysis is generally used for tough-to-lyse cells such as bacteria, fungal, plant/seed, and insect. A “compressive force” will decrease the length of the material on which it acts, such as by squeezing or crushing a material. “Shear force” can cause a material to bend, slide, or twist, such as by sliding one face of the material over an adjacent face.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Characterization of the microbial composition of the two ZymoBIOMICS™ Standards with shotgun metagenomic sequencing (left panel) and 16S rRNA gene targeted sequencing (right panel). The measured composition of the two standards agrees with the theoretical/designed composition. “DNA Standard” represents ZymoBIOMICS™ Microbial Community DNA Standard (DNA version) and “Microbial Standard” represents ZymoBIOMICS™ Microbial Community Standard (cellular version). Genomic DNA composition by shotgun sequencing was calculated based on counting the amounts of raw reads mapped to each genome. 16S composition by 16S rRNA gene targeted sequencing was calculated based on counting the amount of 16S raw reads mapped to each genomes.

FIGS. 2A-2B: (A) Lysis Efficiency of mixed beads (0.1 mm and 0.5 mm) compared to 0.5 mm using a stool sample in varying amounts: (100, 200, and 400 μl). DNA quantified via Nanodrop. (B) The lysis efficiency of bead beating materials (glass beads, zirconium oxide, garnet beads and ultra-dense ceramic beads) was evaluated using Listeria monocytogenes and Saccharomyces cerevisiae as a model of tough to lyse organisms.

FIGS. 3A-3C: Visual evaluation of various bead beating matrices for lysis of Saccharomyces cerevisiae including (A) ZR BashingBead Lysis Tube (Microbe), (B) Mo Bio PowerLyser Lysis Tube (0.1 mm Glass Beads), and (C) Mo Bio Power Lysis Tubes (0.7 mm Garnet).

FIGS. 4A-4B: (A) Using a (high speed disruptor) MP FastPrep 24, the effect of bead size on lysis efficiency was evaluated using Listeria monocytogenes and Saccharomyces cerevisiae. (B) Using a (low speed disruptor) Vortex Genie, the effect of bead size on lysis efficiency was evaluated using Listeria monocytogenes and Saccharomyces cerevisiae.

FIG. 5: Evaluation of varying bead volumes with a constant ratio of 1:1 of beads (0.5 mm and 0.1 mm).

FIGS. 6A-6B: (A) Bead bashing of Listeria monocytogenes using various ratios of bead sizes shown in the graph as a ratio of 0.5 mm/0.1 mm with a constant volume of 600 ul total beads. Nanodrop results displayed in data graph and gel electrophoresis to confirm quantification data and DNA quality. (B) Bead bashing of Saccharomyces cerevisiae using various ratios of bead sizes shown in the graph as a ratio of 0.5 mm/0.1 mm with a constant volume of 600 ul total beads. Nanodrop results displayed in data graph and gel electrophoresis to confirm quantification data and DNA quality.

FIGS. 7A-7B: Benchmarking DNA extraction processes with ZymoBIOMICS™ Microbial Community Standard. The ZymoBIOMICS™ DNA Mini Kit provides unbiased representation of the organisms extracted from the ZymoBIOMICS™ Microbial Community Standard. (A) Comparison of theoretical microbial composition to ZymoBIOMICS™ kit, HMP protocol, Supplier M, and Supplier Q. (B) Comparison of ZymoBIOMICS™ to chemical lysis.

FIG. 8: There is a significant increase in yield and Gram-positive abundance when DNA was isolated using the ZymoBIOMICS™ DNA Mini Kit. The ZymoBIOMICS™ DNA Mini Kit reliably isolates DNA from even the toughest to lyse gram positive organisms, enabling unbiased analyses of microbial community compositions.

FIGS. 9A-9B: (A) Bead bashing ZymoBIOMICS™ Microbial Community Standard bead sizes shown in the graph as a ratio of 0.5 mm/0.1 mm (with a constant volume of 600 μl) in comparison to 600 μl of 0.5 mm beads only. Nanodrop results of DNA yield displayed in data graph. (B) Bead bashing ZymoBIOMICS™ Microbial Community Standard using various ratios of bead sizes shown in the graph as a ratio of 0.5 mm/0.1 mm (with a constant volume of 600 μl) in comparison to 600 μl of 0.5 mm beads only. All samples subject to 16s rRNA sequencing. Ratio of different bacteria given as colors shown by the corresponding legend.

FIG. 10: Time titration of bead beating Bacillus subtilis using the Fisher Scientific Advanced 96 Plate Vortex Shaker Plate.

FIG. 11: Yield comparison of bead beating for different cell types using the Fisher Scientific Advanced 96 Plate Vortex Shaker Plate.

FIGS. 12A-12C: (A) Position legend labelling what position the lysis tubes were placed within the 96-Well Deep Well block, diagonal chosen for variation in position of the tubes. (B) Yield of DNA as measured by Nanodrop spectrophotometry from samples lysed at different locations throughout a 96-well Deep Well Block. (C) Analysis of DNA by agarose gel electrophoresis.

FIGS. 13A-13C: (A) Analysis of DNA yield from bead bashing lysis efficiency time trial of Saccharomyces cerevisiae at 20 and 40 minutes. (B) Analysis of DNA yield from bead bashing lysis efficiency time trial of Listeria monocytogenes at 20 and 40 minutes. (C) Agarose gel electrophoreses of DNA isolate in lysis efficiency time trial.

FIGS. 14A-14B: (A) Position legend labelling what position the lysis tubes were placed within the 96-Well Deep Well block, diagonal chosen for variation in position of the tubes. (B) Percentage of bacterial strain compositions identified from DNA isolated using different methods. All samples were subject to 16s rRNA sequencing. Ratio of different bacteria given as colors shown by the corresponding legend.

FIGS. 15A-15B: (A) Comparison of yield of bead bashing using MP FastPrep 24. Listeria monocytogenes (Lm) and Saccharomyces cerevisiae (Sc) were both used. Using either lysis solution or GLB as a bead beating solution. (B) Comparison of yield of bead bashing using Fisher Scientific Advanced 96 Plate Bead bashing device on Lm and Sc using either Lysis solution or GLB as a bead beating solution.

FIGS. 16A-16D: (A) Bead bashing in Lysis solution vs. GLB with Listeria monocytogenes pure culture. Time points 1, 2, and 3 minutes on the MP FastPrep 24 at 6.5 m/s. Verified via Nanodrop and Gel electrophoresis. (B) Bead bashing in Lysis solution vs. GLB with Listeria monocytogenes pure culture. Time points 20, 35, 50 minutes were tested via Fisher Scientific Advanced 96 Plate Bead bashing device. Verified via Nanodrop and Gel electrophoresis. (C) Bead bashing in Lysis solution vs. GLB with Saccharomyces cerevisiae pure culture. Time points 1, 2, and 3 minutes on the MP FastPrep 24 at 6.5 m/s. Verified via Nanodrop and Gel electrophoresis. (D) Bead bashing in Lysis solution vs. GLB with Saccharomyces cerevisiae pure culture. Time points 20, 35, 50 minutes were tested via Fisher Scientific Advanced 96 Plate Bead bashing device. Verified via Nanodrop and Gel electrophoresis.

FIGS. 17A-17B: Plot of average samples percentage yield for mechanical lysis of (A) Listeria and (B) Sacharomyces cerevisiae cells. Each chart shows an identical sample pattern from stationary, 8 mm, and 15 mm offset test runs. Each chart shows a point plot (solid line) and a corresponding linear trending (dotted line) plot for each of the tests.

FIGS. 18A-18B: (A) Average percent yield vs. sleeve-to-tube gap at 4000 rpm. (B) Percent yield vs. bead load in 2 mL tubes at 4000 rpm.

FIGS. 19A-19B: (A) 48-well plate spacing test (Cryp, list, and Sac): high-yield bashing is only with the perimeter wells due to lower magnetic coupling at the middle. (B) 48-well plate spacing test (Cryp, List, and Sac): high-yield bashing for List and Sac; Cryp yields are marginal-to-high.

FIGS. 20A-20E: (A) Percent yield for each indicated bashing magnet shape. (B) Schematic of source well and target well. (C-E) RT-PCR analysis for indicated tubes.

FIGS. 21A-21D: (A) Schematic depicting orientation of bashing magnet and drive magnet. (B) Schematic depicting orientation of bashing with lateral oscillation. (C) Schematic for configuration of automatic liquid handler. (D) Schematic for configuration for standalone manual lysing.

### DESCRIPTION OF ILLUSTRATIVE EMBODIMENTS

Certain embodiments of the present disclosure provide method and devices for the unbiased release of nucleic acids from mixed microbial samples and efficient lysis of cell cultures for preferred assay sensitivity. Specifically, an automated protocol can comprise the use of a bench-top device that can apply a mechanical force to vessels comprising a mixed microbial sample and high density beads. The method and device can concurrently perform cell lysis in a plurality of sample containers by mechanical cell agitation. The method may be compatible with a broad array of microbial targets including tough-to-lyse microbes. Particularly, the combined effect of the applied force and the beads provide a unique environment that allows for lysis of a wide range of microbial cells leading to lysis that is less biased towards easy to lyse cells such gram-negative bacteria. The high density beads can be comprised of different sizes, and preferably comprise a mixture of at least two differently sized beads to provide for the efficient lysis of microbial samples. Nucleic acids, specifically genomic DNA, can be isolated from soil, microbial fermentation, water, biofilms, and/or eukaryotic cellular cultures or biological body fluids (e.g. sputum, feces, lymph fluid, cerebrospinal fluid (CSF), urine, serum, sweat, various aspirates, and other liquid biological sources) and solid tissues. In particular, the sample may comprise one or more different organisms, such as bacteria including gram negative and gram positive bacteria, archaea, fungi, spores, protozoans, single cell and multicellular parasites, oocyst and algae or other encapsulated or bound nucleic acids. The isolated nucleic acids may be used for microbiome or metagenome analyses.

### I. AUTOMATED METHOD FOR NUCLEIC ACID ISOLATION

Certain embodiments of the present disclosure provide methods, particularly automated methods, and devices for the isolation of nucleic acids from mixed microbial samples. The microbial samples, such as bacterial cells, may be lysed to obtain a pure form (i.e., a cleared bacterial or cellular lysate). In some embodiments, the method comprises the steps of disposing the plurality of samples into an array of sample containers (e.g., each containing a plurality of beads) and applying force to the sample containers, wherein each sample container comprises the sample and a bead for disrupting microbial cells or spores or other types of cells (e.g., bacteria, fungi, spores, protozoans) and viruses, thereby providing an unbiased release of microbial nucleic acids. The method may comprise obtaining samples of one or more organisms, contacting the sample with a plurality of beads and a lysis buffer, and applying a sheer force to lyse the sample and release the nucleic acids (e.g., DNA and/or RNA). Further, the supernatant comprising the nucleic acids may be separated from the plurality of beads and the nucleic acids are then purified from the supernatant using methods known in the art.

In some embodiments, the method comprises adding a plurality of samples into an array of sample containers and applying a mechanical force to the sample container. The sample containers may comprise the sample, a bashing magnetic bar, and a bead or a plurality of beads for disrupting the sample, such as microbial cells and viruses. Accordingly, the present methods can provide an unbiased release of microbial nucleic acids.

In certain aspects, the device for applying an automated mechanical force to the bashing magnetic bar, and consequently to the beads, may comprise a source of a spinning magnetic field below the sample container and an oscillating drive plate that oscillates the sample container in a horizontal plane. Accordingly, also provided herein is a device comprising a driving magnetic field source placed beneath the sample container. The sample container may be placed on the mechanical drive plate which is capable of lateral or orbital oscillation in relation to the magnetic field source. The magnetic field source may be comprises of a spinning magnet around a vertical axis, wherein the magnet polarity is normal to the magnet spinning axis.

The center positions of the drive plate and sample container array may be aligned with the spinning axes of the drive magnet. The drive plate oscillation velocity may be in the range of the amount of the individual sample container cross section size over 1 to 30 seconds. The displacement of the drive plate may be in the range of 0.5 to 2.5 of the individual sample container cross section size.

The mechanical force may be set by the controller. The mechanical force and its distribution across the array of sample containers may be a function of spinning magnet velocity and linear displacement and its rate of the drive plate. Mechanical force applied to the plurality of bashing magnetic bars may be produced by the interaction between magnetic poles of the bashing magnetic bar and the magnetic poles of the spinning magnet below the sample container. The stirring and tumbling action of the bashing magnetic bar can translate applied mechanical energy into the mechanical bashing movement inside of each sample container.

The mechanical agitation of the beads inside the sample container can be achieved by multiple methods. In one method, an external spinning magnetic field source may provide lateral or orbital oscillation to a bashing magnetic bar in each sample container. In another method, external mechanical oscillation may be applied to the sample container. The mechanical oscillation may be lateral oscillation, vertical oscillation, orbital oscillation, or a combination thereof. This method can be performed without a bashing magnetic bar. The device for applying the mechanical force to the sample container or stirring bar array may comprise a shaker. In an alternate method, the external oscillation may be applied to a stirring bar array comprises of a plurality of bashing probes presented into a corresponding array of the sample container. This method may also be performed without a bashing magnetic bar. The device for applying the mechanical force to the sample container or stirring bar array may comprise a shaker. For example, the shaker may be, but is not limited to, a Teleshake 1536-6 microplate shaker (Veriomag).

The device may be constructed from non-magnetic materials, such as non-magnetic aluminum, stainless steel, Delrin, or Mu-metal. The device may have a hard-anodized surface. The screws may be non-magnetic stainless steel and a thread locker, such as Loctite, may be applied to the screws. The drive magnet may have dimensions of about 0.1-10 inches, such as 1, 2, 3, 4, 5, 6, 7, 8, or 9 inches, particularly a size of 2 inch×2 inch×2 inch. Alternatively, multiple magnets may be used of a smaller size, such as 2 inch×2 inch×0.5 inch. The spin velocity of the magnet may be about 1000-10,000 rpm, such as 2000, 3000, 4000, 5000, 6000, 7000, 8000, or 9000 rpm. The plate carrier may be compatible with standard plates, such as a 96-well, 48-well, or 24-well plate. The lateral movement of the plate carrier may have an offset of about 5-50 mm, such as about +/−10-20, 15-25, 20-30, or 25-35 mm. The rate of the lateral movement may be about 0.1-10 repetitions per minute, such as 0.5-2 repetitions per minute.

In some aspects, the device may have manual controls, such as a magnet drive motor with an rpm regulator and/or a titer plate carrier motor with an rpm regulator. The device may have a ready or running indicator light. The device can have one or more automated processes or commands such as, but not limited to, start sample propagation cycle-operator interface, start buffer dispensing, start sample dispensing, sample propagation cycle completed, ready to remove titer plate, start sample bashing, sample bashing is completed, and/or start sample processing.

Further provided herein is a kit comprising a plurality of high density beads, a bashing magnetic bar, a device for applying an automated mechanical force via a spinning magnetic field source to bashing magnetic bar, and a control samples. In some aspects, the control sample comprises a mixed microbial population having a known proportion of each microbial component.

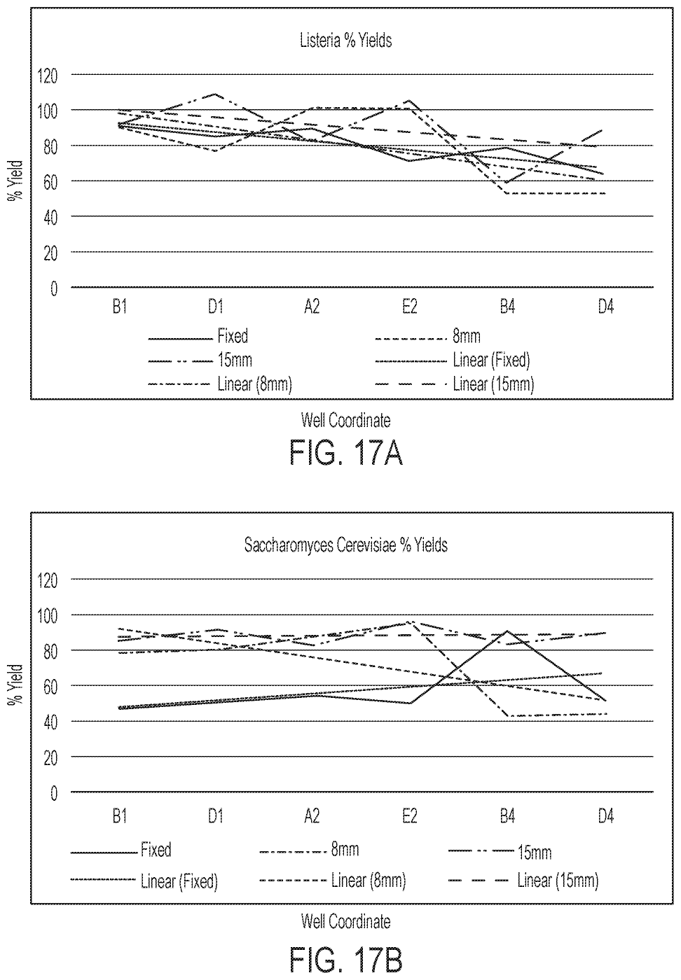

In some embodiments, the automated protocol may use workstations such as the Microlab STAR™ line or the Biomek 4000 automated workstation to apply a force to samples comprising beads. Components of the workstations may include a Deep Well Block, shaker (e.g., the Hamilton® Heater Shaker), and a sample homogenizer or bead beating device (e.g., the MP Biomedicals Fastprep sample homogenizer). Further bead beating devices, shakers, vortex mixers, and or similar that may be used with the present disclosure are listed in FIGS. 17A-17B. Exemplary bead bashing devices include the MP-Bio FastPrep-24, the MP-Bio FastPrep-96, the 2025 Geno/Grinder, the 2010 Geno/Grinder, TissueLyser LT, and the TissueLyser II (Table 1). Also contemplated herein are devices that are not based on current open platforms. For example, the device may be a closed system that processes a sample to purified nucleic acids. In particular aspects, the device only requires the loading of a cartridge and the bead beating and purification are performed on the platform.

In some aspects, the shaker or bead beating device can be a multi-vessel plate mixer/shaker unit capable of mixing/shaking various samples in multiple vessels simultaneously. For instance, the multi-vessel plate mixer can be a multi-vessel plate vortexer. In general, a shaker is an agitation apparatus directed to agitate a sample, such as a sample in a micro centrifuge tube (e.g., an Eppendorf tube), a centrifuge tube, a vial, or microwell plate. The bead beating device is used to agitate the beads with enough force and frequency to lyse the cell, particularly the tough cell walls of tough to lyse organisms. Cell rupture takes place, when the kinetic energy of the colliding beads exceeds the elastic energy stored in the cell. Inter-bead collision lysis requires relative speeds of ˜0.3 m/s, which could only be achieved at large volumetric chamber vibrations. Inter-bead shear flow to achieve lysis for a typical (Gramm positive) bacteria should be in the range of 5-10 m/s. Consequently inter-beads collisions is the primary mechanism of cell rupture during lysis. In some aspects, shakers can generate an orbital motion for mixing, particularly for shaking, a fluidic sample. In other aspects, the motion of the shaker is horizontal or vertical. For example, a rod can be dipped directly into a tube and agitated. A magnetic stirrer may be used to move a steal object, such as a sphere, within a tube or rack. Grinding motions may also be used as well as sonication and rotar strators.

Exemplary shakers may include the Fisher Scientific Analog Vortex Mixer with a bead bashing tube attachment, the Advanced Microplate Vortex Mixer, the IKA MS3 Digital Vortex, and the Hamilton Heater Shaker module. Generally, shakers can have a speed of about 300 to 5000 rpm, such as 2500 rpm for microtiter platers, up to 200 rpm for deep well plates, or 1800 rpm for tubes. Orbital shakers or other mixing device may be any device suitable for mixing the contents of the tubes, and preferably is able to mix the tubes without removing them from the chamber, such as tube holders. For example, the orbital shakers may be Hamilton Heater Shakers available from Hamilton Robotics of Reno, Nev. The orbital shakers may be operated at room temperature (i.e., no heating), or at a predetermined temperature controlled by heating elements coupled to the shakers (e.g., a resistance coil). U.S. Patent Application No. US20150036450, incorporated herein by reference, discloses a mechanism for generating an orbital motion for shaking a sample accommodated by a sample holder which may be used in the present methods. Other shakers for use in the present disclosure are disclosed in, for example, US2010/218620, U.S. Pat. No. 4,990,130, JP 2007-237036, JP 10-277434, U.S. Pat. No. 6,190,032, EP 1,393797, EP 0,462,257, WO 98/32838, EP 0,679,430, US 2011/286298, U.S. Pat. Nos. 4,990,130, 4,673,297, and US 2009/086573; all incorporated herein by reference. In some aspects, the bead beating or bead moving device is as described in U.S. Pat. Nos. 7,495,090, 5,702,950, 6,942,806, 7,622,046, and 8,430,247 as well as International Patent Publication No. WO1996012959.

The array of sample containers (e.g., a plurality of beads) may be comprised within a chamber. The chamber can be a container of any suitable material, size, and shape. In certain embodiments, the chamber is a plastic container. The chamber may, for example, be in the shape of a micro centrifuge tube (e.g., an Eppendorf tube), a centrifuge tube, a vial, microwell plate. The chamber can define only one compartment/chamber for holding cells, beads, and a stir element, or a plurality of discrete compartments/chambers (e.g., an array of wells) for holding mixtures (cells, beads, and stir elements) in isolation from one another. The chamber may be a sealed or sealable container, such as a container with a lid, cap, or cover. The interior surface may be chemically inert to preserve the integrity of the analyte of interest, or the reagents being stored that are necessary for processes such as lysis, binding, washing or eluting a sample.

In certain embodiments, the system further contains a rack configured to hold the chamber. The rack may be configured to hold multiple chambers and can be placed on a support surface for simultaneous processing of multiple samples. The rack may also be used as holder of the chamber for storage purpose. For example, multiple chambers may be placed on the rack and stored in a refrigerator or freezer for further analysis. The chamber may be interfaced with an external instrument (e.g. liquid handling robot, microfluidic device, analytical instrument).

Bead bashing with the high density beads provides rapid, high quality, and unbiased nucleic acids. Bead bashing is one of the preferred methods of sample homogenization and cell lysis method in which a biological sample (e.g. organism, tissue, cell) is agitated (e.g. vigorously agitated) with beads (e.g. glass or other material) to break up the sample and lyse cells through physical means. Beads previously have not been characterized for such an application as uniform unbiased lysis of microbes for accurate community profiling. Higher density beads allow for shorter bead-beating times and result in overall higher yields than the standard and less dense beads. Further, high density beads enable lower speed units to be used which are more suitable for inexpensive highly accessible automation. The mechanical nature of the lysis by bead bashing in conjunction with chemical lysis resulted in improved effectiveness against hard to lyse organisms such as Gram-positive bacteria, archaea, fungi and spores, ensuring that the nucleic acids of all the microbes are obtained in abundances reflecting reality. Current tests evaluate total abundance including dead and alive, however making the distinction between dead and alive has been contemplated and could be achieved using methods known in the art. Combinations of mechanical lysis, enzymatic lysis, and chemical lysis has also been contemplated. The term “lyse” with respect to cells means disruption of the integrity of at least a fraction of the cells to release intracellular components, such as nuclei, nucleic acids and proteins, from the disrupted cells. The term “homogenize” with respect to sample preparation is referring to blending (diverse elements e.g. stool, tissue, sputum, saliva) a sample into a uniform mixture. Both lysis and homogenization are important features for extracting and purifying DNA from a mixed sample that accurately reflects the actual composition. In order to access the proteins or nucleic acid inside of a cell the cell wall must first be ruptured. This process is known as cell lysis. Many techniques exist that can accomplish this including but not limited too chemically, electrically, and mechanically. Mechanical lysis offers various advantages such as being able lyse tough to lyse samples as well as not introducing substances that can affect downstream processes. One such mechanical method is bead bashing in which beads are added to the sample and mechanically excited to cause collisions that lyse the cells.

The rupturing of the cell walls occurs when collisions occur between the beads and the cells. As the beads are mechanically excited by an outside motion collisions will occur. These collisions take the form as compressive or shear interactions as the beads smash the cells against other beads or against the wall of the container. If the energy from these collisions are larger than the elastic energy of the cell walls, then these walls will rupture. The size of the beads can come into play as there needs to be a sufficiently large bead to cause collisions yet small enough that the cells are not simply pushed aside when in the path of the beads. As objects shrink in size their effective viscosity increases. This can cause the cells to simply be pushed aside instead of crushed in the same manner that a leaf floating in water will escape the clutches of your hand as you move your hand through the water trying to grab it. A larger and heavier object will have inertia and the water would simply get pushed over it. But with small objects there is virtually no inertia and the water that is getting pushed takes the leaf with it. As an object gets smaller and small (such as Listeria monocytogenes which is 0.4-2 μm or Saccharomyces cerevisiae is 5-10 m) the viscous forces start to become dominant. This can be shown with a Reynolds number smaller than 1 (Purcell et al., 1977). When this occurs the fluid is no longer passing over an object but effectively sticking to it. A solution of using multiple diameter beads can be used to solve this issue as well as compensate for various sized organisms and samples. In the addition, the sheer force generated by the beads maybe a contributor to the rupture of the cell walls.

The motion applied to the beads can have an impact on the effectiveness of lysing. Some cyclical motions may not be effective as the beads will fall into an orderly pattern that will not cause many collisions to occur. An example would be when the beads are spun in a circle the beads will all move at the same velocity and same path inside their container. To combat this more chaos should be added to the bead motion. This can be added by moving in a seemingly random motion such as having the samples suspending on springs moving in a non-harmonic motion. Alternatively, by sharply changing the direction of the movement can cause the beads to crash into oncoming beads or the wall of the vessel. An example of this motion would be to raise then lower the beads very rapidly. Careful consideration must be considered for the stroke size because if it is too short and there may not be enough distance for the beads to accelerate while too long could cause the beads to decelerate when switching directions.

The plurality of beads may be made of plastic, glass, ceramic, mineral, metal and/or any other suitable materials. In certain embodiments, the beads may be made of non-magnetic materials. In certain embodiments, the beads are rotationally symmetric about at least one axis (e.g., spherical, rounded, oval, elliptic, egg-shaped, and droplet-shaped particles). In other embodiments, the beads have polyhedron shapes. In some embodiments, the beads are irregularly shaped particles. For example, the beads can be glass, ceramic, silicon (e.g., fumed silica or pyrogenic silica, colloidal silica, silica gel), metal, steel, tungsten carbide, garnet, sand, or sapphire beads.

In some embodiments, the beads have a mean diameter of greater than 1 m (e.g. about 2 μm, about 5 μm, about 10 μm, about 20 μm, about 50 μm, about 100 μm, about 200 μm, about 500 μm, about 1 mm, about 2 mm, about 5 mm, about 1 cm, greater than 1 cm).

In particular embodiments, the plurality of beads (e.g., spherical beads) may comprise beads of different sizes, such as having an average diameter of between 0.01 and 1.0 mm, such as beads of 0.25 and 0.75 mm and beads that are between 0.05 and 0.25 mm in diameter. The beads of different sizes allows for the efficient lysis of the samples. In particular embodiments, the beads comprise a mixture of beads of 0.1 mm and 0.5 mm diameter beads, such as at a ratio of 1:1, 1:2, 1:3, 1:4, 1:5, 1:6 or 2:1, 3:1, 4:1, 5:1, 6:1 by volume. Other mixtures and ratios are contemplated and may be preferred depending on the types of beads used. For instance, a single large steel ball mixed with a plurality of 0.1 mm and 0.5 mm beads to help break down large materials such as tissues. In addition, 2.0-5.0 mm beads maybe used to help break down tissues (e.g. insect, plant, animal). Further 2.0 mm and 0.1 mm represents a preferred embodiment as it is ideal for the disruption of infectious disease carrying vectors (e.g. insects) and the organism they harbor maybe a tough to lyse gram positive bacteria or yeast. In this scenario, the ratio of volume maybe as large as 1:10 (0.1 mm beads and 2.0 mm beads). The types of beads, the size of the beads, and the volume of the tube being filled all affect the volume and ratio of beads used.

To calculate the maximum theoretical force of the beads one would use Newton's 2^{nd} Law of Motion given as:

F=ma (1)

Mass, M, of the beads are known so the acceleration, a, must be solved.

Acceleration is given as a function of the change of velocity, v, over the change of time, t:

$$a = \frac{Δ v}{Δ t} = \frac{{v}_{f} - {v}_{i}}{{t}_{f} - {t}_{i}} \qquad ( 2 )$$

Where the subscripts of f and i correspond with the final and initial states respectively. The initial time will be set to 0 as well as the initial velocity. The initial velocity is presumed to be 0 because the beads are presumed to come to a complete stop before switching directions.

The bead beating machine is presumed to be cyclical and uniform. Such that the stroke length is what determines the final distance and time that the beads travel. Setting the stroke length as S, then the average velocity can be calculated and used as the final velocity as:

$${v}_{f} = \frac{S}{{t}_{f}} \qquad ( 3 )$$

Substituting the equation 3 into the equation 2 and setting the initial values as 0 results in:

$$a = \frac{S}{{t}_{f}^{2}} \qquad ( 4 )$$

Finally combining Equation 1 and 4 results in the force equations:

$$F = m \frac{s}{{t}_{f}^{2}} \qquad ( 5 )$$

This force would be the maximum theoretical possible. There are many factors which could limit the force such as friction from the fluid which would depend on viscosity. Other factrors could potentially include disorderly motion which while may be beneficial to bead beading would prevent the beads from achieving their maximum force. If the beads do not move in a straight line than the effective stroke distance would be smaller and as seen from equation 5 this would reduce the total force.

Using values from Table 1 for the MP-Bio FastPrep-96 as an example then the stroke length is 1.5 in which is 0.0381 m. The time can be calculated from the oscillations of 1500 oscillations a minute. One oscillation is a stroke in one direction and then back to its original position. Converting oscillations a minute to per second is done by multiplying by 1 min/60 sec which results in 25 oscillations per second. Inverting this results in 1/25 or 0.04 seconds per oscillation. Because an oscillation is 2 strokes this can be divided by 2 which is 0.02 seconds per stroke. Therefore the beads can be said to move 0.0381 m in 0.02 seconds. Using this information for equation 5 yields

F=m*0.0381/0.02²=m*95.25 (6)

By plugging in the mass of the beads in newtons will result in the maximum force applied on the beads at ideal conditions in newtons.

Equation 5 can be further expanded by substituting volume and density for mass, m. By pressuming that all the beads are perfect circles this results in:

$$F = \frac{4}{3} π {r}^{3} * ρ * \frac{s}{{t}_{f}^{2}} \qquad ( 6 )$$

Where r is the radius of the sphere and p is the density.

Below are a breakdown of different masses of the beads and different accelerations associated with different machines. The final table is the force of the different beads on the different machines

TABLE 1
Parameters of different bead materials.

| | | Density | |
|---|---|---|---|
| Material | Bead Diameter (m) | (kg/m³) | Mass (kg) |
| Zymo Ceramic (0.1 mm) | 0.0001 | 2100 | 4.189E−12 |
| Zymo Ceramic (0.5 mm) | 0.0005 | 2100 | 5.236E−10 |
| ZrO₂ (0.15 mm) | 0.00015 | 5680 | 1.414E−11 |
| Glass Beads (0.1 mm) | 0.0001 | 2600 | 4.189E−12 |
| Garnet Beads (0.7 mm) | 0.0007 | 3750 | 1.437E−09 |

TABLE 2
Acceleration of different devices.

| | | Stroke | | |
|---|---|---|---|---|
| | Stroke | Time | Acceleration | |
| Device | Distance (m) | (s) | (m/s²) | |
| MP Bio | 0.0381 | 0.02 | 95.25 | |
| FastPrep 96 | | | | |
| Vortex | 0.006 | 0.01875 | 17.067 | Note: The Stroke |
| Mixer | | | | distance was hand |
| | | | | measured off of |
| | | | | taken apart unit |

TABLE 3
Force of the different beads on the different machines

Force (N)

| | MP Bio | |
|---|---|---|
| Material | FastPrep 96 | Vortex Mixer |
| Zymo Ceramic (0.1 mm) | 3.990E−10 | 7.149E−11 |
| Zymo Ceramic (0.5 mm) | 4.987E−08 | 8.936E−09 |
| ZrO₂ (0.15 mm) | 1.347E−09 | 2.413E−10 |
| Glass Beads (0.15 mm) | 3.990E−10 | 7.149E−11 |
| Garnet Beads (0.7 mm) | 1.369E−07 | 2.452E−08 |

TABLE 4
List of exemplary Bead Beating Devices and List of Shakers and Votex
Mixers

| Manufacturer | Name | Bead Size | Material |
|---|---|---|---|
| Zymo | Zymo BashingBead Lysis | 0.1 and 0.5 mm mixed | Ceramic |
| | Tubes (Microbe) | | |
| Zymo | Zymo BashingBead Lysis | 2.0 mm | Ceramic |
| | Tubes (Tissue) | | |
| MoBio | MoBio Garnet Bead Tubes | 0.15 mm | Garnet |
| MoBio | MoBio Garnet Bead Tubes | 0.7 mm | Garnet |
| | (Large) | | |
| MoBio | MoBio Ceramic Bead | 1.4 mm | Ceramic |
| | Tubes | | |
| MoBio | MoBio Ceramic Bead | 2.8 mm | Ceramic |
| | Tubes | | |
| MoBio | MoBio Glass Bead Tubes | 0.5 mm | Glass |
| MoBio | MoBio Glass Bead Tubes | 0.1 mm | Glass |
| MoBio | MoBio Metal Bead Tubes | 2.38 mm | Metal |
| MP-Bio | Lysis Matrix A | Crushed Garent and | Garnet and Ceramic |
| | | ¼″ Ceramic | |
| MP-Bio | Lysis Matrix B | 0.1 mm | Silica |
| MP-Bio | Lysis Matrix C | 1.0 mm | Silica |
| MP-Bio | Lysis Matrix D | 1.4 mm | Ceramic |
| MP-Bio | Lysis Matrix E | 1.4 mm, 0.1 mm, and | Ceramic, Silica, and |
| | | 4.0 mm | Glass |
| MP-Bio | Lysis Matrix F | 1.6 mm and 1.6 mm | Aluminum Oxed and |
| | | | Silicon Carbide |
| MP-Bio | Lysis Matrix G | 1.6 mm and 2.0 mm | Silicon Carbide and |
| | | | Glass |
| MP-Bio | Lysis Matrix H | 2.0 mm and 2.0 mm | Glass and Yellow |
| | | | Zirconium |
| MP-Bio | Lysis Matrix I | 2.0 mm and 4.0 mm | Yellow Zirconium and |
| | | | Black Ceramic |
| MP-Bio | Lysis Matrix J | 2.0 mm and 1.6 mm | Yellow Zirconia and |
| | | | Aluminum Oxide |
| MP-Bio | Lysis Matrix K | 0.8 mm | Zirconium Silicate |
| MP-Bio | Lysis Matrix M | 6.35 mm | Zirconium Oxide |
| MP-Bio | Lysis Matrix S | 3.175 mm | Stainless Steel |
| MP-Bio | Lysis Matrix SS | 5.5 mm | Stainless Steel |
| MP-Bio | Lysis Matrix Y | 0.5 mm | Yttria-Stabalized |
| | | | Zirconium Oxide |
| MP-Bio | Lysis Matrix Z | 2.0 mm | Yttria-Stabalized |
| | | | Zirconium Oxide |
| Qiagen | Stainless Steel Beads | 5.0 mm | Stainless Steel |
| Qiagen | Stainless Steel Beads 2 | 7.0 mm | Stainless Steel |
| Qiagen | Tungsten Carbide Beads | 3.0 mm | Tungsten Carbide |
| Qiagen | Pathogen Lysis Tubes S | N/A | Glass (?) |
| Qiagen | Pathogen Lysis Tubes L | N/A | Glass (?) |

A list of exemplary Bead Beating Matrices is provided below

### MP-Bio FastPrep24

### Capacity—(24) 2 ml Tubes

Time—1-60 seconds, programmable with 1 second increments
Speed—4-6.5 m/s, programmable with 0.5 m/s increments
Acceleration—<2 seconds to maximum speed
Dimensions—270 mm×425 mm×330 mm

### MP-Bio FastPrep96

Capacity—(2) 96 well plates
Time—100-300 seconds
Speed—1800 oscillations/min at a 1.5 inch stroke

### 2025 Geno/Grinder

### Capacity—(48) 96 Well Plates

Time—120 seconds
Speed—500-1750 strokes/min at a 1.25 inch stroke

### Dimensions—12.88″×18.88″×35.88″

### 2010 Geno/Grinder

### Capacity—(6) 96 Well Plates

Time—120 seconds
Speed—500-1750 strokes/min at a 1.25 inch stroke

### Dimensions—15″×20.49″×25.25″

### TissueLyser LT

Capacity—(12) 2 ml microcentrifuge tubes
Time—40-300 seconds
Speed—1800 oscillations/min
Dimensions—150 mm×270 mm×280 mm

### TissueLyser II

### Capacity—(2) 96 Well Plates

Time—15-180 seconds
Speed—1800 oscillations/min
Dimensions—Not available

### List of Shakers and Vortex Mixers:

Fisher Scientific Analog Vortex Mixer w/ 2 ml bead bashing tube attachment
Capacity—(24) 2 ml tubes
Time—1200 seconds

### Speed—3200 RPM

Dimensions—12.2 cm×17.3 cm×12.2 cm

### Advanced Microplate Vortex Mixer

### Capacity—(1) 96 Well Plates

Time—3600 seconds

### Speed—3500 RPM

Dimensions—26.7 cm×13.7 cm×11.4 cm

### IKA MS3 Digital Vortex

### Capacity—(1) 96 Well Plates

Time—3600 seconds

### Speed—3000 RPM

Dimensions—148 mm×63 mm×205 mm

### Hamilton Heater Shaker

### Capacity—(1) 96 Well Plates

Time—3600 seconds

### Speed—2500 RPM

In particular embodiments, the plurality of beads and sample are contacted with a lysis buffer (e.g., containing a buffering agents, chaotropic salts, ionic detergents, non-ionic detergents solvents, EDTA, Trizol, monovalent and divalent salts). In some embodiments, the present disclosure provides appropriate salts (e.g. NaCl, KOH, MgCl₂, etc.) and salt concentration (e.g. high salt, low salt, 1 mM, 2 mM, 5 mM, 10 mM, 20 mM, 50 mM, 100 mM, 200 mM, 500 mM, 1 M, 2M, 3M, 4M, 5M, etc.) for use with the array of sample containers (e.g., a plurality of beads). In some embodiments, buffers for use with the array of sample containers (e.g., a plurality of beads) may include, but are not limited to H₃PO₄/NaH₂PO₄, Glycine, Citric acid, Acetic acid, Citric acid, MES, Cacodylic acid, H₂CO₃/NaHCO₃, Citric acid, Bis-Tris, ADA, Bis-Tris Propane, PIPES, ACES, Imidazole, BES, MOPS, NaH₂PO₄/Na₂HPO₄, TES, HEPES, HEPPSO, Triethanolamine, Tricine, Tris, Glycine amide, Bicine, Glycylglycine, TAPS, Boric acid (H₃BO₃/Na₂B₄O₇), CHES, Glycine, NaHCO₃/Na₂CO₃, CAPS, Piperidine, Na₂HPO₄/Na₃PO₄, and combinations thereof.

As indicated above, DNA such as genomic DNA can be isolated from one or more cells, bodily fluids or tissues. An array of methods can be used to isolate DNA from samples such as swabs, blood, sweat, tears, lymph, urine, saliva, semen, cerebrospinal fluid, amniotic fluid, feces, soil, water, sludge, etc. DNA can also be obtained from one or more cell or tissue in primary culture, in a propagated cell line, a fixed archival sample, forensic sample or archeological sample. The sample may be a “host derived sample” referred to herein as any organism that serves as an environment for microorganisms to reside on whether it resides as a resident or as a transient. Animals and plants frequently serve as such hosts for microorganisms. Methods for isolating genomic DNA from a cell, fluid or tissue are well known in the art (see, e.g., Sambrook et al., 2001). Yeast species (e.g. Saccharomyces cerevisiae), fungi species, other microorganisms, human (Homo sapiens) liquid tissue (e.g. sputum, lymph fluid, cerebrospinal fluid (CSF), urine, serum, sweat, various aspirates, and other liquid biological sources) solid tissue, or tissue from a variety of species commonly used in diagnostic, research or clinical laboratories are contemplated as compatible with this purification procedure as sources of DNA and are all alternative embodiments of the present invention. The bacterial species may comprise gram positive or gram negative strains, such as one or more of the strains Bacillus subtilis, Listeria monocytogenes, Staphylococcus aureus, Enterococcus faecalis, Lactobacillus fermentum, Salmonella enterica, Escherichia coli, Pseudomonas aerupinosa, Saccharomyces cerevisiae, and Cryptococcus neoformans. Procedures for handling and preparing samples from these various species are well known in the art and are reported in the scientific literature. However, methods for isolating nucleic acids simultaneously from a single sample containing a mixed population of organisms and potentially including host tissue or cells such as human has not been described. Mechanical lysis has been described as the most robust method for such extraction (liberation of nucleic acids) from such a diverse range of easy to lyse and tough to lyse cells due to its stochastic nature, however actual validations of this method did not exist until very recently. The ZymoBIOMICS Microbial Community Standard is the first commercially available standard to enable such analyses. Using this Mock Microbial Community Standard, we found that the most cited methods, which presumed to be free of significant bias, strongly overrepresented gram negative organisms.

In certain embodiments, the present methods further comprise the purification and analysis of the DNA and/or RNA released from the sample using sheer or compression or tensile forces. The further analysis may comprise, for example, microbiome or metagenome analyses, PCR, arrays, 16S rRNA gene sequencing, and shotgun sequencing.

Isolation of DNA and RNA is well known in the art. In particular embodiments, DNA isolation is performed using a commercially available kit such as the ZymoBIOMICS™ DNA Mini Kit. In particular aspects, the isolation is performed free of PCR inhibitors, such as polyphenols, humic and fulvic acids). In exemplary methods, plasmid isolation comprises modified mild alkaline lysis of host cells containing a plasmid, sodium hydroxide (NaOH) and sodium dodecyl sulphate (SDS), NaOH/SDS, denaturation, and precipitation of unwanted cellular macromolecular components as an insoluble precipitate, coupled to column-based silica, or other chromatography or purification methods. Isolation buffers based on alkaline lysis protocols are well known in the art and variations of compositions are contemplated as embodiments of the present invention that are compatible with various commercially available chromatographic columns and technologies. Alkaline lysis procedures generally use sodium acetate, potassium acetate, as well as a variety of other salts, including chaotropic salts. Ribonuclease RNAase A is commonly added to degrade contaminating RNA from the lysate. The clarification of the lysate can be performed by centrifugation or filtration methods both of which are known in the art. The plasmid is pure, typically with an OD260/280 ratio above 1.8. The plasmid DNA is suitably pure for use in the most sensitive experiments.

A number of methods have been used to isolate DNA from samples. For example, U.S. Pat. No. 5,650,506 relates to modified glass fiber membranes which exhibit sufficient hydrophilicity and electropositivity to bind DNA from a suspension containing DNA and permit elution of the DNA from the membrane. The modified glass fiber membranes are useful for purification of DNA from other cellular components. U.S. Pat. Nos. 5,705,628 and 5,898,071 disclose a method for separating polynucleotides, such as DNA, RNA and PNA, from a solution containing polynucleotides by reversibly and non-specifically binding the polynucleotides to a solid surface, such as a magnetic microparticle. A similar approach has been used in a product, “DYNABEADS DNA Direct” marketed by DYNAL A/S, Norway. Similarly, glass, plastic and other types of beads have been used to bind to and isolate DNA from solutions. Commercially, ZymoResearch offers the ZymoBIOMICS™-96 MagBead DNA Kit which includes beads for homogenization of diverse samples.

In addition to adaptation of mechanical homogenization onto existing nucleic acid purification platforms, one of the preferred embodiments is a device in which the homogenization and lysis is integrated into a dedicated purification system built for purpose. The device would incorporate mechanical lysis, in one of its various forms previously described, wherein bead beating is one of the preferred the methods, followed by purification of the nucleic acids from the lysate. The mechanical lysis method employed would efficiently lyse a range of organisms such that the resulting nucleic acids purified are not significantly biased towards a specific organism enabling accurate community profiling and or characterization of nucleic acids found within a sample. The purification method used could be based on methods previously described, but not limited to said methods, wherein a preferred embodiment utilizes chaotropes and/or alcohols to induce reversible binding of nucleic acids to a mineral matrix such as silica or magnetic silica beads. If using a mineral matrix, the sample may be passed through the matrix using various methods common in the art such positive or negative pressure using for instance syringes, pumps, vacuums, or centrifuges. Magnetic beads maybe manipulated by various means including moving them using a rod as previously described or liquid handling approaches where the beads are held in place and liquids are transferred.

In some aspects, the nucleic acid is isolated as described by Ruggiere et al. (Springer Protocols Handbooks, Sample Preparation Techniques for Soil, Plant, and Animal Samples, 41-52, 2016; incorporated herein by reference). For example, phase separation techniques utilizing phenol-chloroform or acid guanidinium thiocyanate-phenol-chloroform extraction (e.g., Tri-Reagent® or Trizol® by commercial suppliers MRC and Invitrogen, respectively) and column-based separation techniques (that use a solid phase carrier such as silica or anion exchange resins) are the most prevalent methods used for nucleic acid isolation. Other technologies have also been employed for the binding and purification of nucleic acid including nitrocellulose, polyamide membranes, glass particles (powder or beads), diatomaceous earth, and anion-exchange materials (such as diethylaminoethyl cellulose).

Organic phase extraction of nucleic acids involves adding phenol and chloroform to a sample. The result is the formation of a biphasic emulsion which, upon centrifugation, the organic-hydrophobic solvents containing lipids, proteins, and other cellular components will settle on the bottom of the aqueous layer that contains the nucleic acids (Kirby, 1956; Grassman & Deffner, 1953; Tan & Yiap, 2009). The aqueous phase is subsequently partitioned from the organic layer for use in the precipitation of the nucleic acids. Ethanol (or isopropanol) with ammonium acetate (or some ionic salt) is used to precipitate the nucleic acids from the partitioned aqueous layer (Tan & Yiap, 2009). The nucleic acid is pelleted by centrifugation, washed with ethanol, and then resuspended in the desired low-salt solution (usually water or TE) for use in downstream analysis.

Due to the inherent nature of the chemistry of organic separation, DNA and RNA can be co-purified or selectively isolated individually. To selectively isolate DNA, an RNase A treatment may be necessary to remove RNA present in the aqueous layer (Rogers and Bendich, 1985). For effective DNA isolation, the aqueous layer must have a basic pH. Acidification using acid guanidinium thiocyanate-phenol-chloroform extraction, forces DNA to be partitioned into the interphase and organic phase, allowing for convenient isolation of RNA directly from the aqueous phase (Chomczynski & Sacchi, 1987 and Chomczynski et al., 1989).

In column-based separation, such as silica-based methods, use of a chaotropic agent, such as guanidinium chloride, will cause nucleic acids to selectively (and reversibly) bind to silica particles. The silica-nucleic acid-bound complexes can be subsequently washed with an alcohol solution to remove contaminants and then the nucleic acids eluted using water or TE. Spin-column extractions are well characterized and highly consistent due to reduced handling compared to phenol-chloroform extractions (Price et. al., 2009). They allow for quick and efficient purification by circumventing many of the problems associated with organic-phase separation such as incomplete phase separation and hassle of working with highly toxic solvents (Tan & Yiap, 2009).

1. RNA Purification Method

Several methods are available for the purification of RNA, such as described above. For example, the Zymo Quick-RNA™ MiniPrep Plus kit may be used to purify high-quality total RNA. In addition, Zymo DNA/RNA Shield™ ensures nucleic acid stability during sample storage/transport at ambient temperatures. In one exemplary method, RNA may be purified by the methods described in U.S. Pat. No. 9,051,563, incorporated herein by reference. In general, the method comprises (a) obtaining sample comprising a nucleic acid molecule and phenol and (b) contacting the sample to a silica substrate in the presence of a binding agent comprising a chaotropic salt, an alcohol or a combination thereof, thereby binding the nucleic acid molecule to the silica substrate. In certain aspects, a nucleic acid containing sample may comprise a substantial amount of phenol, such as about or greater than about 10%, 20%, 30%, 40% or 50% phenol by volume. A binding agent may comprise an alcohol such as a lower alcohol, e.g., methanol, ethanol, isopropanol, butanol or a combination thereof.

The addition of a chaotropic salt may be used for cell lysis and the formation of an RNA-containing precipitate. The term chaotropic salt refers to a substance capable of altering the secondary or tertiary structure of a protein or nucleic acid, but not altering the primary structure of the protein or nucleic acid. Examples of chaotropic salts include, but are not limited to, guanidine thiocyanate, guanidine hydrochloride sodium iodide, potassium iodide, sodium isothiocyanate, and urea. Guanidine salts other than guanidine thiocyanate and guanidine hydrochloride may be used as a chaotropic salts in the subject methods. Preferred chaotropic salts for use in the present methods are guanidine hydrochloride and guanidine thiocyanate. The concentration of chaotropic salt used to elicit RNA-containing precipitant formation may vary in accordance with the specific chaotropic salt selected. Factors such as the solubility of the specific salt must be taken into account. Routine experimentation may be used in order to determine suitable concentration of chaotropic salt for eliciting RNA-containing precipitate formation. In embodiments of the present methods employing guanidine hydrochloride as the chaotropic salt, the concentration of guanidine hydrochloride in the nucleic acid containing solution from which the RNA-containing precipitate is obtained is in the range of 1 M to 3 M, 2 M being particularly preferred. In embodiments of the present methods employing guanidine thiocyanate as the chaotropic salt, the concentration of guanidine thiocyanate in the nucleic acid-containing solution from which the RNA-containing precipitate is obtained is in the range of 0.5 M to 2 M, 1 M being particularly preferred. Combinations of chaotropic salts may be used to elicit RNA-containing precipitate formation. In embodiments of the invention employing multiple chaotropic salts, the chaotropic salts may be added in the form of concentrated solution or as a solid (and dissolved in the initial RNA-containing preparation).

After the addition of the chaotropic salts, the solution is allowed to incubate for a period of time sufficient to permit an RNA-containing precipitate to form. Unless the incubation conditions are modified during incubation, e.g., a temperature change, the longer the period of incubation time, the larger the quantity of RNA precipitate that will form. Incubation preferably occurs under constant temperature conditions. When a sufficient quantity of RNA precipitate for the purpose of interest, e.g., cDNA library formation, is formed, the RNA precipitate may be collected. The quantity of RNA precipitate formed may be monitored during incubation. Monitoring may be achieved by many methods, such methods include visually observing the formation of the precipitate (e.g., visually), collecting the precipitate during the incubation process and the like. In most embodiments of the invention, incubation time is at least one hour, preferably incubation is at least eight hours. Periods for incubation may be considerably longer than eight hours; no upper limit for incubation time is contemplated although need to obtain isolated RNA in a reasonable amount of time may be a constraint.

The temperature of the mixture formed by adding the chaotropic salt to the RNA-containing composition of interest, e.g., mixed microbial sample, influences the amount of RNA-containing precipitate formed in the subject method. In general, a greater precipitate yield will be obtained at a lower temperature, i.e., below room temperature. Preferably, freezing is avoided; however, a RNA-containing precipitate may form if a fresh cellular lysate is rapidly frozen. Additionally, lower temperatures may be used to reduce the activity of RNAses or detrimental chemical reactions occurring in the processed sample. Preferably, the temperature of the solution from which the RNA-containing precipitate formed is in the range of 1° C. to 25° C., more preferably in the range of 4° C. to 10° C.

After the RNA-containing precipitate has formed, the RNA-containing precipitate is collected. Collection entails the removal of the RNA-containing precipitate from the solution from which the precipitate was formed. The precipitate may be separated from the solution by any of the well-known methods for separation of a solid phase from a liquid phase. For example, the RNA-containing precipitate may be recovered by filtration or centrifugation. Many types of filtration and centrifugation systems may be used to collect the RNA-containing precipitate. Precautions against RNA degradation should be taken during the RNA precipitate collection step, e.g., the use of RNAase-free filters and tubes, reduced temperatures.

After the RNA-containing precipitate has been recovered, the precipitate may optionally be washed so as to remove remaining contaminants. A variety of wash solutions may be used. Wash solutions and washing conditions should be designed so as to minimize RNA losses from the RNA-containing precipitate. Preferably a wash solution containing the same chaotropic salt used to form the RNA-containing precipitate is used to wash the collected RNA-containing precipitate. The concentration of the chaotropic salt in the wash solution is preferably high enough for an RNA-containing precipitate to form, thereby minimizing losses of the RNA-containing precipitate during the washing process. Additionally, the washing solution is preferably at a temperature sufficiently low for RNA-containing precipitates to form, thereby minimizing losses of the RNA-containing precipitate during the washing process.

The collected RNA-containing precipitate may be solubilized so as to enable subsequent manipulation of the purified RNA in solutions. Solubilization may be accomplished by contacting the collected RNA-containing precipitate with a solution that does not elicit the formation of an RNA-containing precipitate. Typically, such a solution is an aqueous buffer (low ionic strength) or water. Examples of such buffers includes 10 mM Tris-HCl (pH 7.0), 0.1 mM EDTA; suitable buffering agents include, but are not limited to, tris, phosphate, acetate, citrate, glycine, pyrophosphate, aminomethyl propanol, and the like. The RNA-containing precipitate and the solution may be actively mixed, e.g., by vortexing, in order to expedite the solubilization process.

2. Magnetic Bead DNA Purification of Nucleic Acids

In some embodiments, the nucleic acids are purified using magnetic microparticles, such as magnetic beads. Silica materials, including glass particles, such as glass powder, silica particles, and glass microfibers prepared by grinding glass fiber filter papers, and including diatomaceous earth, have been employed in combination with aqueous solutions of chaotropic salts to separate nucleic acids from other substances and render the nucleic acids suitable for use in molecular biological procedures (see U.S. Pat. No. 5,075,430; incorporated herein by reference). Such matrices are designed to remain bound to the nucleic acid material while the matrix is exposed to an external force such as centrifugation or vacuum filtration to separate the matrix and nucleic acid material bound thereto from the remaining media components. The nucleic acid material is then eluted from the matrix by exposing the matrix to an elution solution, such as water or an elution buffer. Numerous commercial sources offer silica-based matrices designed for use in centrifugation and/or filtration isolation systems. See, e.g. Wizard™ DNA purification systems line of products from Promega Corporation (Madison, Wis., U.S.A.); or the QiaPrep™ line of DNA isolation systems from Qiagen Corp. (Chatsworth, Calif., U.S.A.). Exemplary magnetic particle purification methods are disclosed in, for example, U.S. Pat. Nos. 6,027,945, 6,284,470, 6,673,631, and 7,078,224; each incorporated herein by reference.

A complex of the silica magnetic particles and the DNA target material is formed by exposing the particles to the medium containing the DNA target material under conditions designed to promote the formation of the complex. The complex is preferably formed in a mixture of the silica magnetic particles, the medium, and a chaotropic salt. The complex is removed from the mixture using a magnetic field. Other forms of external force in addition to the magnetic field can also be used to isolate the biological target substance according to the methods of the present invention after the initial removal step. Suitable additional forms of external force include, but are not limited to, gravity filtration, vacuum filtration and centrifugation. The nucleic acid material is eluted from the silica magnetic particle by exposing the complex to an elution solution. The elution solution is preferably an aqueous solution of low ionic strength, more preferably water or a low ionic strength buffer at about a pH at which the nucleic acid material is stable and substantially intact. Any aqueous solution with an ionic strength at or lower than TE buffer (i.e. 10 mM Tris-HCl, 1 mM ethylenediamine-tetraacetic acid (EDTA), pH 8.0) is suitable for use in the elution steps of the present methods, but the elution solution is preferable buffered to a pH between about 6.5 and 8.5, and more preferably buffered to a pH between about 7.0 and 8.0. TE Buffer and distilled or deionized water are particularly preferred elution solutions for use in the present invention. The low ionic strength of the preferred forms of the elution solution described above ensures the nucleic acid material is released from the particle. Other elution solutions suitable for use in the present methods will be readily apparent to one skilled in this art.

Chaotropic salts are salts of chaotropic ions. Such salts are highly soluble in aqueous solutions. The chaotropic ions provided by such salts, at sufficiently high concentration in aqueous solutions of proteins or nucleic acids, cause proteins to unfold, nucleic acids to lose secondary structure or, in the case of double-stranded nucleic acids, melt (i.e., strand-separate). It is thought that chaotropic ions have these effects because they disrupt hydrogen-bonding networks that exists in liquid water and thereby make denatured proteins and nucleic acids thermodynamically more stable than their correctly folded or structured counterparts. Chaotropic ions include guanidinium, iodide, perchlorate and trichloroacetate. Preferred in the present invention is the guanidinium ion. Chaotropic salts include guanidine hydrochloride, guanidine thiocyanate, sodium iodide, sodium perchlorate, and sodium trichloroacetate.

At least two commercial silica magnetic particles are particularly preferred for use in the present disclosure, BioMag® Magnetic Particles from PerSeptive Biosystems, and the MagneSil™ Particles available from Promega Corporation (Madison, Wis.). Any source of magnetic force sufficiently strong to separate the silica magnetic particles from a solution would be suitable for use in the nucleic acid isolation methods of the present invention. However, the magnetic force is preferably provided in the form of a magnetic separation stand, such as one of the MagneSphere® Technology Magnetic Separation Stands (cat. nos. Z5331 to 3, or Z5341 to 3) from Promega Corporation.

When the target nucleic acid is genomic DNA, it is necessary to disrupt the tissue to release the target genomic DNA from association with other material in the tissue, so the target genomic DNA can adhere to the pH dependent ion exchange matrix in the presence of a solution at the first pH. The resulting complex of matrix and genomic DNA is separated from the disrupted tissue, and washed to remove additional contaminants (if necessary). The genomic DNA is then eluted from the complex by combining the complex with an elution solution having a second pH which is higher than the first pH.

Magnetic microparticles useful in the present method can be a variety of shapes, which can be regular or irregular; preferably the shape maximizes the surface areas of the microparticles. The magnetic microparticles should be of such a size that their separation from solution, for example by filtration or magnetic separation, is not difficult. In addition, the magnetic microparticles should not be so large that surface area is minimized or that they are not suitable for microscale operations. Suitable sizes range from about 0.1μ mean diameter to about 100μ mean diameter. A preferred size is about 1.0μ mean diameter. Suitable magnetic microparticles are commercially available from PerSeptive Diagnostics and are referred to as BioMag COOH (Catalog Number 8-4125).

As used herein, the term “magnetic particles” or “magnetic microparticles” refers to materials which have no magnetic field but which form a magnetic dipole when exposed to a magnetic field, i.e., materials capable of being magnetized in the presence of a magnetic field but which are not themselves magnetic in the absence of such a field. The term “magnetic” as used in this context includes materials which are paramagnetic or superparamagnetic materials. The term “magnetic”, as used herein, also encompasses temporarily magnetic materials, such as ferromagnetic or ferrimagnetic materials with low Curie temperatures, provided that such temporarily magnetic materials are paramagnetic in the temperature range at which silica magnetic particles containing such materials are used according to the present methods to isolate biological materials.

Salts which have been found to be suitable for binding DNA to the microparticles include sodium chloride (NaCl), lithium chloride (LiCl), barium chloride (BaCl₂), potassium (KCl), calcium chloride (CaCl₂)), magnesium chloride (MgCl₂) and cesium chloride (CeCl). In one embodiment sodium chloride is used in the present of PEG or cationic detergents such as CTAB. The wide range of salts suitable for use in the method indicates that many other salts can also be used and can be readily determined by one of ordinary skill in the art. Yields of bound DNA decrease if the salt concentration is adjusted to less than about 0.5M or greater than about 5.0M. The salt concentration is preferably adjusted to about 1.25M. In one embodiment, the magnetic microparticles with bound DNA are washed with a suitable wash buffer solution before separating the DNA from the microparticles by washing with an elution buffer. A suitable wash buffer solution has several characteristics. First, the wash buffer solution must have a sufficiently high salt concentration (i.e., has a sufficiently high ionic strength) that the DNA bound to the magnetic microparticles does not elute off of the microparticles, but remains bound to the microparticles. Suitable salt concentrations are greater than about 1.0M and is preferably about 5.0M. Second, the buffer solution is chosen so that impurities that are bound to the DNA or microparticles are dissolved. The pH and solute composition and concentration of the buffer solution can be varied according to the type of impurities which are expected to be present. Suitable wash solutions include the following: 0.5×5 SSC; 100 mM ammonium sulfate, 400 mM Tris pH 9, 25 mM MgCl₂ and 1% bovine serum albumine (BSA); and 5M NaCl. A preferred wash buffer solution comprises 25 mM Tris acetate (pH 7.8), 100 mM potassium acetate (KOAc), 10 mM magnesium acetate (Mg₂ OAc), and 1 mM dithiothreital (DTT). The magnetic microparticles with bound DNA can also be washed with more than one wash buffer solution. The magnetic microparticles can be washed as often as required to remove the desired impurities. However, the number of washings is preferably limited to two or three in order to minimize loss of yield of the bound DNA. Yields of DNA when the microparticles are used in excess are typically about 80% after washing with a wash buffer and eluting with an elution buffer.

An affordable automated purification device with bead beating integrated onto the device for unbiased nucleic acid extraction by multiple methods can be developed comprising the aforementioned methodologies and the following is an example of a preferred embodiment. The device could utilize bead mover technology as exemplified by the Maxwell® 16 Purification System or KingFisher™ Flex Purification System for the purification of nucleic acids from a lysate using chaotropic salts and magnetic silica microparticles. Other chemistries and methods of purification would be apparent to one skilled in the art and could readily be substituted such as but not limited to PEG/Carboxylated Microparticles or PEG/Cellulose Microparticles. The purification would be performed in a cartridge composed of plurality of wells that could be filled with a plurality of components for the nucleic acid isolation and purification including, but not limited to bead beating beads for lysis, magnetic silica beads for purification, bead beating solutions, binding solutions, washing solutions, and elution solutions. In a preferred case, the bead beating solution and binding solution are the same solution. The wells would either be sufficiently deep such that during the bead beating process no cross contamination occurs or a sealing mechanism such as a foil or plunger is used to prevent cross contamination. The sample would be loaded into a well containing the lysis solution and BashingBeads. The bead beating could be performed using an orbital shaker or modified orbital shakers to provide a more chaotic motion which is preferable to increase the number of collisions. The speed at which the unit moves would be between 1,000 RPM and 10,000 RPM. More preferably the speed would be between 2,000 RPM and 5,000 RPM. The most preferred speed would be one in which an unbiased lysis occurs as validated using a mock microbial community standard such as the ZymoBIOMICS Microbial Community Standard which includes a range of organisms of varying recalcitrance to lysis (i.e. yeast, gram-positive bacteria, and gram-negative bacteria). An alternative approach would be that the bead mover executes an agitation motion that causes the bead bashing as opposed to the plate where the cartridge is inserted. A preferred embodiment would be a system where the minimal force is met to lyse cells of plurality of sizes and recalcitrance to lysis wherein “tough-to-lyse” cells as efficiently lysed as the “easy-to-lyse cells” such that reasonably uniform lysis is achieved and the number of collisions per second is sufficient to lyse them in a reasonable period of time (i.e. <1 hour and more preferably <20 minutes) at a speed and motion that is achievable in an affordable manner. Following lysis, if binding reagents are already present the bead mover could transfer the binding beads into the well with the lysate and shake gently to facilitate homogenous binding without breaking down the binding beads. A preferred speed for such operations is below 1,000 RPM and more preferably below 500 RPM. If the lysis solution did not contain binding reagents a dispenser such attached to a simple pump such as, but not limited to, a peristaltic pump to dispense a binding reagent. One contemplated embodiment is parallel purification of DNA and RNA from the same sample and in this case, it may be preferable to add alcohol via a separate dispenser unit and have two sets of magnetic binding beads in order to achieve parallel purification of DNA and RNA. Following binding the magnetic beads would be transferred using the bead mover to the next well(s) containing wash buffers where mixing preferably occurs, but is not required, and finally transferred to the elution buffer where mixing preferably occurs but is also not required. Lastly, the magnetic beads are transferred out of the solution so that the users may remove the eluate. In some instances, the final well maybe a tube that can be removed such as a 1.5 ml centrifuge tube.

### II. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Automated Nucleic Acid Purification System

Microbial Community Standard: With the idea of fully automated purification system that incorporates unbiased and lysis (i.e., bead beating) directly on a device that is capable of extracting nucleic acids from complex samples containing mixed populations of organisms of varying recalcitrance and that the extracted nucleic acids accurately reflect the actual nucleic acid profile present within the sample it quickly became clear that a mock microbial community would be required to create, optimize and validate the system. This led to the development of the ZymoBIOMICS Microbial Community Standard (Tables 4-5) encompassing 10 organisms (2 yeast), 5 gram positive bacteria, and 3 gram negative bacteria. The yeast and gram positive organisms are generally considered to be tough to lyse and the gram negative organisms are generally considered relatively easy to lyse. Saccharomyces cerevisiae and Listeria monocytogenes were specifically included in the standard due their known recalcitrance to lysis. Using the ZymoBIOMIC Microbial Community Standard extraction accuracy or bias could be determined by representation of the organisms present post sequencing the sample using 16s rRNA Gene Sequencing or Shotgun Metagenomic Sequencing. When all other parameters except extraction methods are held constant the over or under representation of organisms could be directly correlated to the ability of the method to efficiently or at least equally lyse the ten organisms in the standard of varying sizes and cell wall composition/hardiness.

TABLE 4
List of Organisms and their theoretical mixed abundance.

| Species | GC % | Gram Stain | gDNA Abun. (%) |
|---|---|---|---|
| Pseudomonas aeruginosa | 66.2 | − | 12 |
| Escherichia coli | 56.8 | − | 12 |
| Salmonella enterica | 52.2 | − | 12 |
| Lactobacillus fermentum | 52.8 | + | 12 |
| Enterococcus faecalis | 37.5 | + | 12 |
| Staphylococcus aureus | 32.7 | + | 12 |
| Listeria monocytogenes | 38.0 | + | 12 |
| Bacillus subtilis | 43.8 | + | 12 |
| Saccharomyces cerevisiae | 38.4 | Yeast | 2 |
| Cryptococcus neoformans | 48.2 | Yeast | 2 |

TABLE 5
Composition of the standard is highly accurate and free of
contamination as determined using the mOTU method.
Microbial composition was profiled with shotgun metagenomic
sequencing (178 million reads). Taxonomy identification was
performed with mOTU (bork.embl.de/software/mOTU/).

| Species | mOTU counts | mOTU Abun. (%) |
|---|---|---|
| Bacillus subtilis | 9048 | 11.86 |
| Listeria monocytogenes | 11454 | 15.01 |
| Staphylococcus aureus | 7960 | 10.43 |
| Enterococcus faecalis | 11322 | 14.84 |
| Lactobacillus fermentum | 17081 | 22.39 |
| Salmonella enterica | 7939 | 10.41 |
| Escherichia coli | 6994 | 9.17 |
| Pseudomonas aeruginosa | 4484 | 5.88 |
| Propionibacterium acnes | 1 | 0.0013 |

Bead Selection: Material and Size (0.5 mm Vs. Mixed [0.1 mm and 0.5 Mm]):

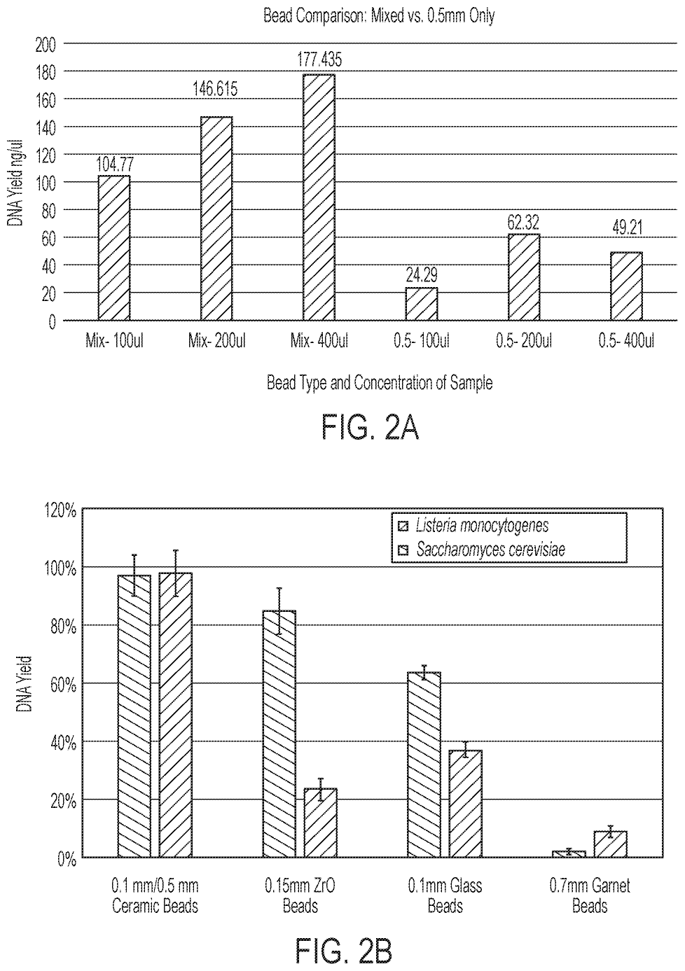

Mixed beads (0.1 mm and 0.5 mm) versus 0.5 mm beads was evaluated using increasing quantities of stool. The 0.5 mm beads were filled into 2 ml screw cap tubes to a volume of 600 l and the 0.1 mm and 0.5 mm mixed beads were filled to a volume of 300 μl each respectively. Varying range of stool inputs were processed (100 μl, 200 μl, and 400 μl) to determine the effect on yield. Bead beating was performed using the individual tube format lysis with MP FastPrep 24 at 6.5 m/s for 5 minutes. Each sample was then removed from the beads and purified using the ZymoBIOMICS DNA Miniprep kit protocol (D4300). Each sample was analyzed using a Nanodrop to determine the yield. It was found that the mixed beads method was the most effective at lysing stool (FIG. 2). Four bead beating matrices were evaluated (ultra-dense ceramic beads, zirconium oxide, glass beads, and garnet beads) for their ability to rupture (lyse) microbial cell walls to release nucleic acids. The model organisms chosen were Listeria monocytogenes and Saccharomyces cerevisiae due to their recalcitrance to lysis and distinctly different size and cell wall composition. Bead beating was performed on the MP FastPrep 24 (high speed disruptor) at 6.5 m/s for a duration of 5 minutes. After bead beating, the lysate was purified using the ZymoBIOMICS DNA Miniprep Kit. Lysis efficiency was measured in the context of yield. 100% yield was determined by using Lysozyme (to lyse Listeria monocytogenes) and Zymolyase (to lyse Saccharomyces cerevisiae) followed by bead beating and Proteinase K digestion prior to purification using the ZymoBIOMICS DNA Miniprep Kit. This combination represented the most complete form of lysis and liberation of DNA. (FIG. 2B) The proprietary mixed (0.1 mm and 0.5 mm; ratio 1:1) ultra-dense ceramic beads achieved essentially 100% of the expected DNA while the other bead types struggled with either one of both of the model organisms tested. Zirconium oxide beads effectively lysed Listeria monocytogenes (approximately 85% recovery 23%), but struggled with the Saccharomyces cerevisiae. Glass beads achieved 63% of Listeria monocytogenes and 35% Saccharomyces cerevisiae. Garnet beads performed very poorly with 2% recovery of Listeria monocytogenes and 9% recovery of Saccharomyces cerevisiae. The observations from this study indicated the importance of selecting the correct bead type for maximizing yield, sensitivity, and reducing bias. Only the mixed ultra-dense ceramic beads enabled close to 100% efficient lysis and outlined the type of beads should continue being used moving forward to achieve unbiased lysis.

Visual Evaluation of Various Bead Beating Matrices for Lysis of Saccharomyces cerevisiae:

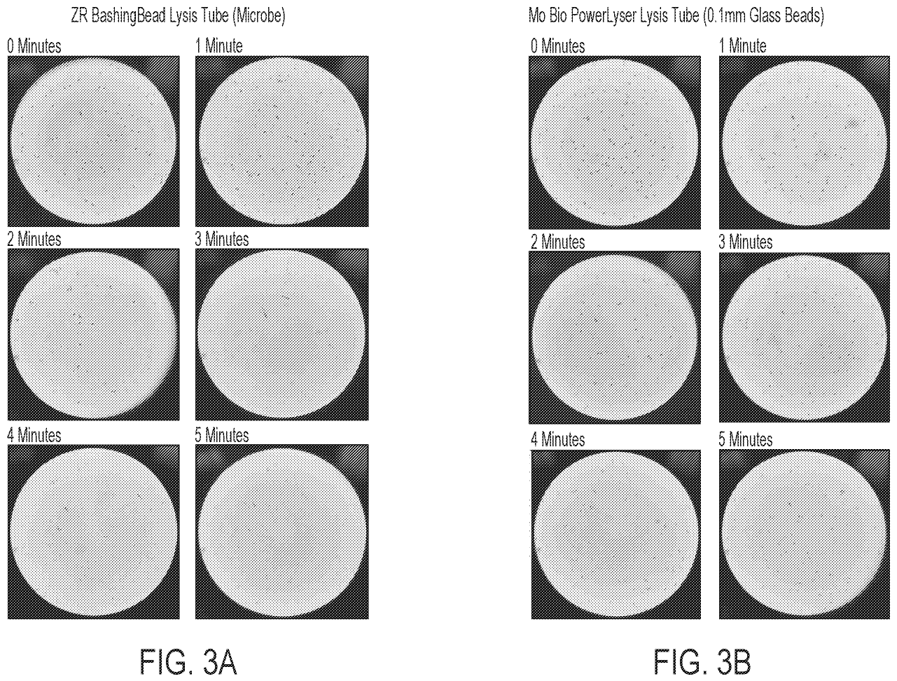

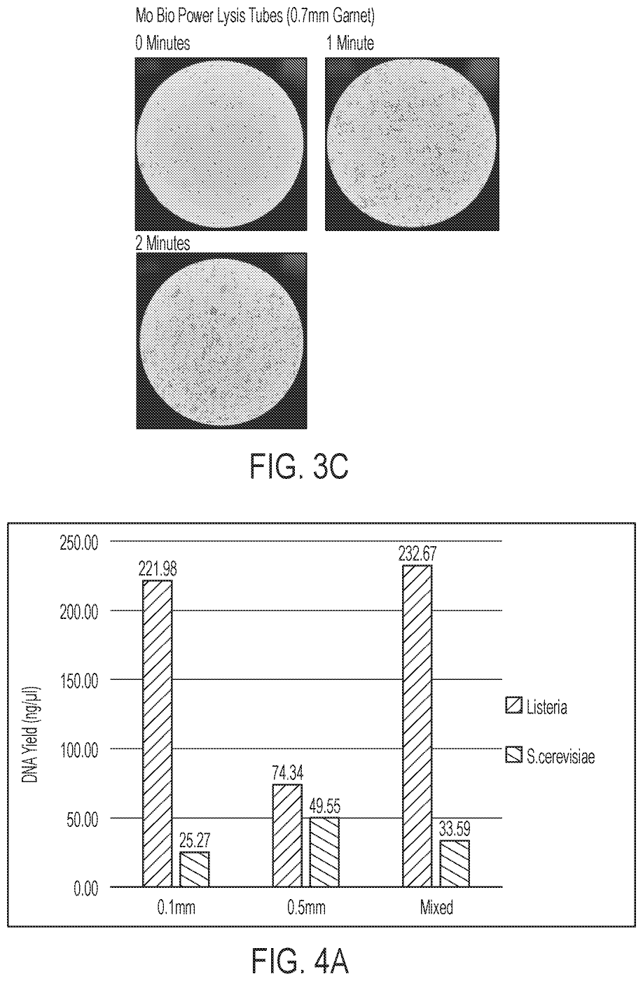

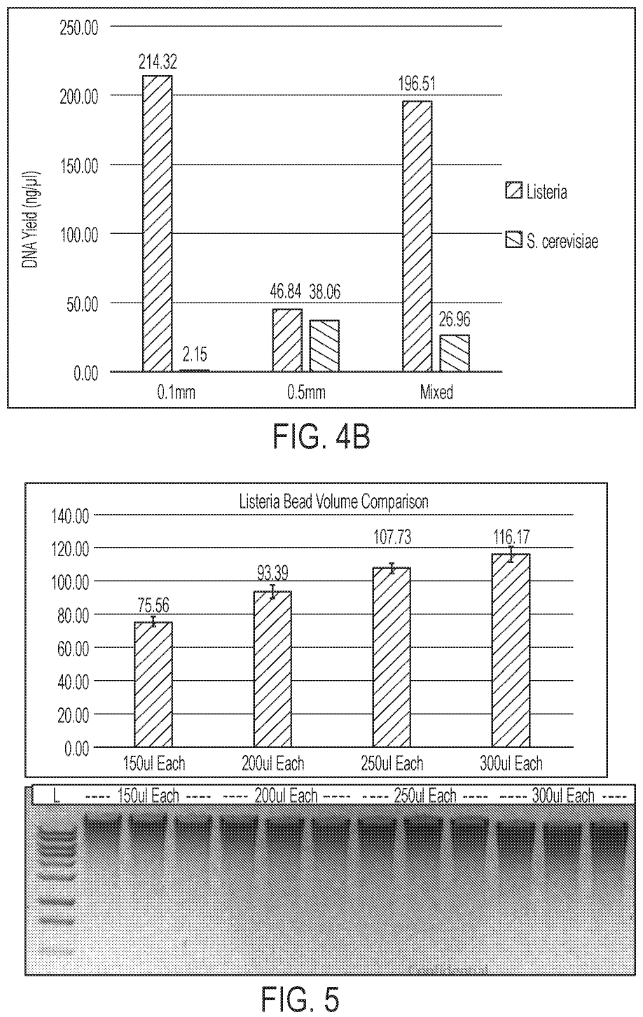

Saccharomyces cerevisiae was used as the model tough organism compare three different lysis matrices. Lysis ability of these bead bashing matrices were evaluated visually using a microscope at 40× amplification for evaluation of remaining intact cells. Lysing matrices used are as follows: ultra-dense 0.1 mm and 0.5 mm ceramic beads, garnet beads, and glass beads. 150 μl of S. cerevisiae culture mixed with 700 μl of PBS buffer was mixed and added to the individual lysis tubes. 2 μl of sample was added to a glass slide and pictures were taken through the microscope for documentation. Bead bashing was performed using the MP FastPrep 24. All groups were checked at 0, 1, 2, 3, 4, and 5-minute time points to assess how effectively cells were lysed. The data from these visual experiments agreed well with the results from the extraction experiment. The mixed beads effectively lysed the Saccharomyces cerevisiae while the glass beads and garnet beads did not effectively lyse these cells (FIG. 3).

High Speed and Low Speed Disruptors and the Effect of Bead Size on Lysis:

High speed and low speed disruptor units were evaluated for lysis efficiency of microbes in the context of different bead sizes. The model organisms were Listeria monocytogenes and Saccharomyces cerevisiae due to their widely recognized recalcitrance to lysis and distinctly size and cell wall composition were used to evaluate high speed and low speed disruptors and the effect of bead size on lysis. Bead beating was performed on the MP FastPrep 24 (high speed disruptor) at 6.5 m/s for a duration of 5 minutes and the Vortex Genie (low speed disruptor) with a horizontal 2 ml lysis tube adapter for 20 minutes. After bead beating, the lysate was purified using the ZymoBIOMICS DNA Mini Kit. The type of beads used in this experiment were Zymo's proprietary ultra-dense ceramic beads. The bead groups tested were 1) 0.1 mm bead only (600 μl total bead volume), 2) 0.5 mm bead only (600 μl total bead volume), 3) 0.1 mm and 0.5 mm beads (600 μl total bead volume; ratio 1:1). All three bead groups were evaluated using both disruptor devices and organisms. When using the MP FastPrep 24, Listeria monocytogenes was lysed most effectively when using mixed beads (0.1 mm and 0.5 mm) and 0.1 mm ceramic beads. The 0.5 mm beads alone did not effectively lyse the small and robust Listeria monocytogenes even when using the high-speed disruptor indicating force and the number of collisions was not the limiting factor influencing the lysis of the Listeria monocytogenes. Instead, the rupture of the Listeria monocytogenes cell walls by the 0.5 mm was poor because of the fact that as objects shrink in size their effective viscosity increases leading to low numbers of actual collisions. The 0.1 mm beads alone using the high-speed disruptor also lead to approximately 50% reduced lysis efficiency as compared to the 0.5 mm beads. The most likely cause of this observation is that force exerted by the small beads is inadequate for efficient lysis. In the case of low speed disruptor, (Vortex Genie with adapter) the 0.1 mm beads were completely ineffective at lysing the yeast organisms. The most likely cause of this is also due to the force exerted being completely inadequate. While the much heavier 0.5 mm beads are capable of exerting enough force even when using a low speed disruptor. The mixed beads again facilitate the most robust and versatile lysis across both sample types and disruptor units. Therefore, the novel combination of 0.1 mm and 0.5 mm ultra-dense ceramic beads for robust lysis of highly diverse microbial organisms enables the use of low speed disruptors which taken together is an enabling technology for the development of affordable fully automated integration of bead beating integration with nucleic acid purification.

Effect of Bead Volume on Yield:

To optimize lysis efficiency there is a preferred volume of beads. A constant ratio of beads was used throughout this experiment (1:1 ratio of 0.5 mm and 0.1 mm beads), but the total volume of beads was varied. A culture of Listeria monocytogenes was grown and pelleted. The cell pellet was suspended in 500 μl of water and each purification used 50 μl. Beadbeating was performed in the 2 ml screw cap tube using the MP FastPrep 24 at 6.5 m/s at a duration of 5 minutes. All conditions were held constant except the volume of beads used. The groups included Group 1: 300 μl of beads (150 μl each), Group 2: 400 μl of beads (200 each), Group 3: 500 μl of beads (250 μl each), and Group 4: 600 μl of beads (300 μl each). Each lysate was processed using the ZymoBIOMICS DNA Mini Kit (D4300). Each sample was then quantified using Nanodrop and electrophoresed using a 1% agarose gel (FIG. 5). It was observed that the total volume of beads is critical to lysis efficiency. There was a direct correlation between the total yield and the quantity of beads used and therefore the number of collisions that can occur in a tube over a fixed duration of time.

Effect of Changing the Bead Ratio:

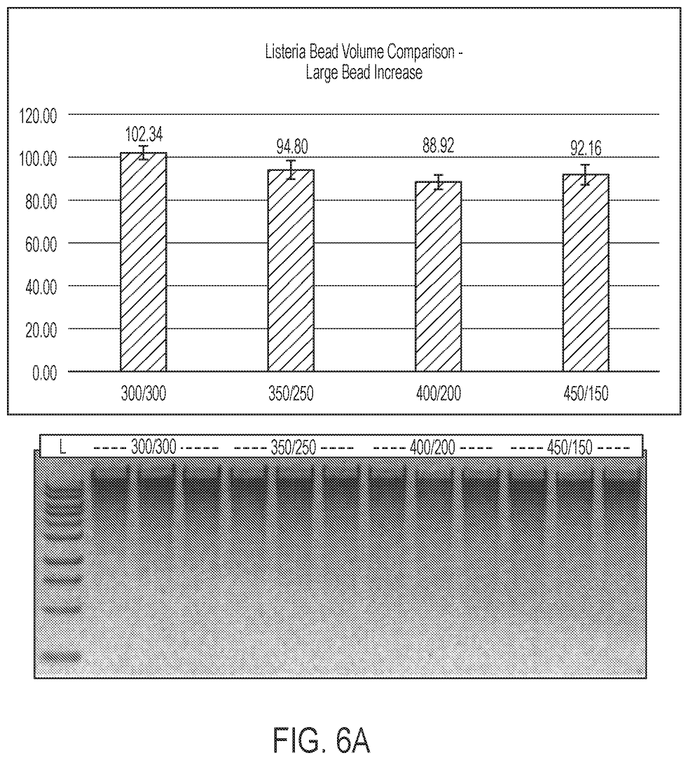

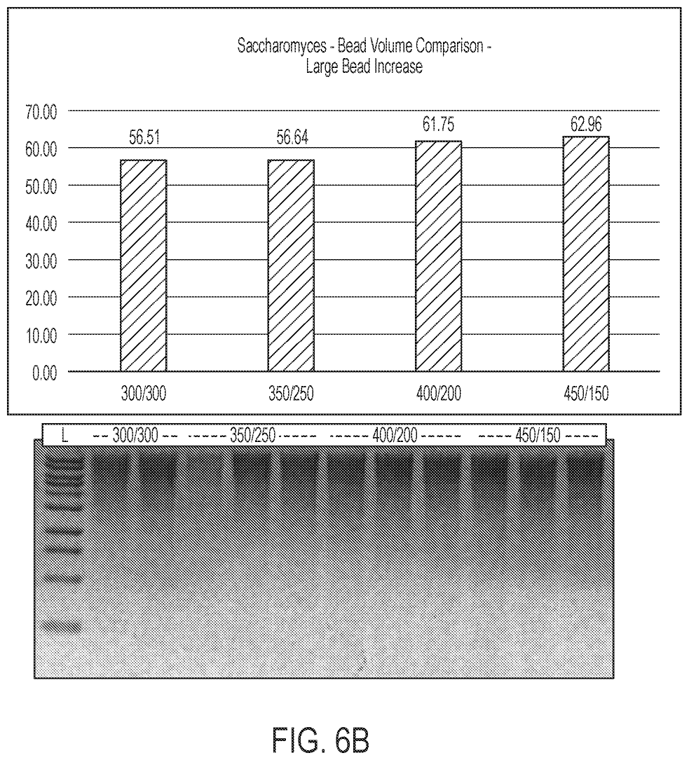

Listeria monocytogenes and Saccharomyces cerevisiae were cultured and pelleted. After suspension in water 50 μl of Listeria monocytogenes and 500 μl of Saccharomyces cerevisiae, were used to assess the effect varying ratios of mixed beads (0.1 mm and 0.5 mm). Experiments were performed using the MP FastPrep 24 at 6.5 m/s for 5 minutes. All conditions were held constant except the ratio of beads used. The groups included Group 1: Control (300/300-0.5 mm/0.1 mm), Group 2: 350/250-0.5 mm/0.1 mm, Group 3: 400/200-0.5 mm/0.1 mm, and Group 4: 450/150-0.5 mm/0.1 mm. Each sample was purified using the ZymoBIOMICS DNA Mini Kit (D4300). Each sample was then quantified using Nanodrop and electrophoresed using a 1% agarose gel (FIGS. 6A-B). Changing the ratio of beads appeared to result in small changes in the yield of both Listeria monocytogenes and Saccharomyces cerevisiae.

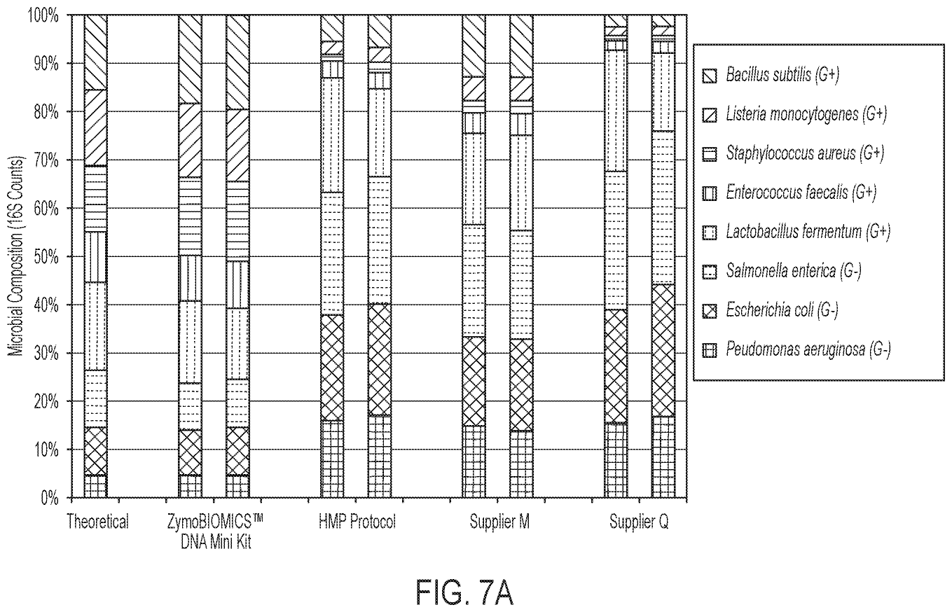

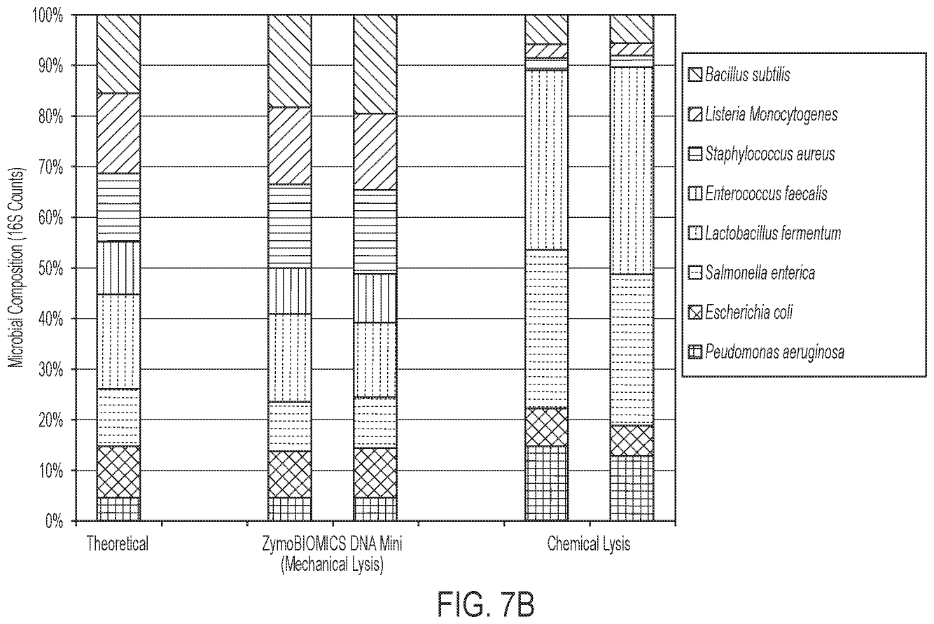

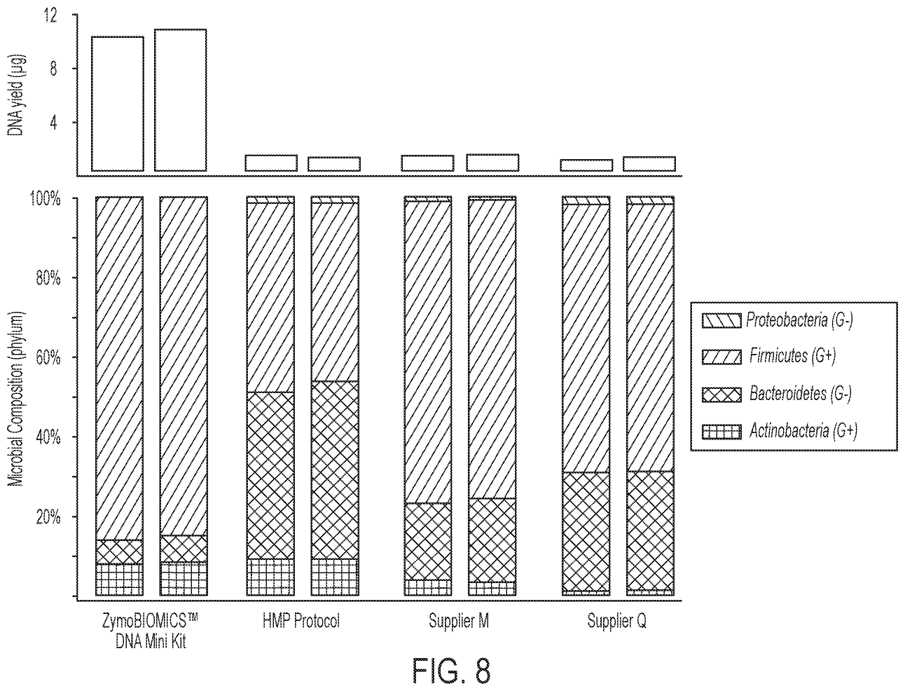

The Mock Microbial Community Standard was used to evaluate the effect of bead ratio on lysis bias. The ZymoBIOMICS Microbial Community Standard was used to evaluate varying ratios of beads which were subsequently purified using the ZymoBIOMICS DNA Minprep Kit. In addition, the two most cited extraction methods were evaluated (PowerSoil® DNA Isolation Kit and the Human Microbiome Project Protocol). Each method was evaluated using 100 μl of ZymoBIOMICS Microbial Community Standard. Bead Beating was performed on the MP FastPrep 24 at 6.5 m/s for a duration of 5 minutes. Each kit or method was performed according to the recommended protocol and all other conditions were held constant. The ratio of 0.5 mm beads to 0.1 mm beads were as follows: Group 1: (300/300-0.5 mm/0.1 mm), Group 2: 350/250-0.5 mm/0.1 mm, Group 3: 400/200-0.5 mm/0.1 mm, and Group 4: 450/150-0.5 mm/0.1 mm. Each sample was quantified using Nanodrop Samples were analyzed using 16S rRNA gene sequencing. 16S rRNA genes were amplified with primers targeting v3-4 region and the amplicons were sequenced on Illumina® MiSeq™ (2×250 bp). Overlapping paired-end reads were assembled into complete amplicon sequences. The composition profile was determined based on sequence counts after mapping amplicon sequences to the known 16S rRNA genes of the eight different bacterial species. The ZymoBIOMICS Community Standard is composed of a diverse range of organisms including both tough and easy to lyse and therefore is useful to identify biases in lysis efficiency. The ratio of beads was shown to have little effect on bias, however the absence of the 0.1 mm beads did lead to bias further indicating the need for mixed beads (FIGS. 7A-7B). The PowerSoil® DNA Isolation Kit and the Human Microbiome Project Protocol were both significantly biased towards gram negative organisms indicating poor lysis efficiency even when using high powered bead beater units such as the MP FastPrep 24. If the power was substantially decreased the number and force of collisions may also be drastically reduced further compromising the quality of data achieved using these methods (Beckers et al., 2010).

Bias Free Microbial Extraction Using Mixed Beads (0.1 mm and 0.5 Mm) ZymoBIOMICS DNA Mini Kit:

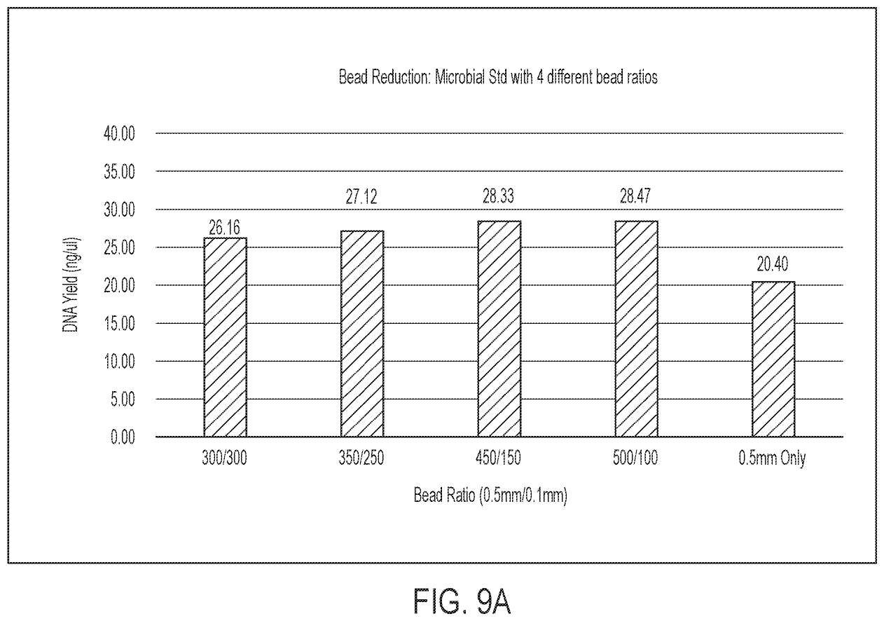

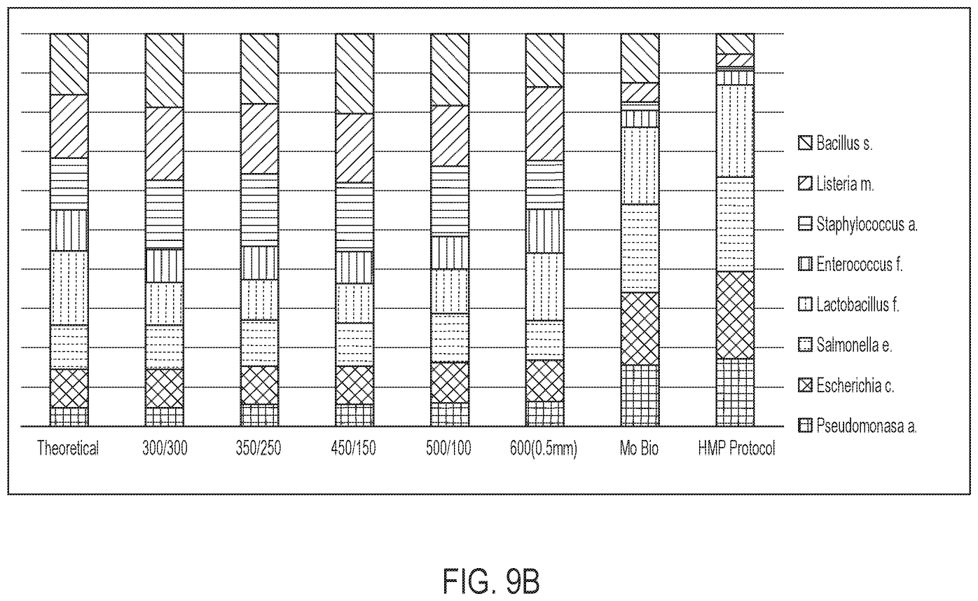

DNA was extracted from ZymoBIOMICS™ Microbial Community Standard using four different DNA extraction methods (ZymoBIOMICS™ DNA Mini Kit, Human Microbiome Project Protocol, PowerSoil DNA Isolation Kit, and QIAamp DNA Stool Mini Kit) and analyzed using 16S rRNA gene sequencing. In addition, to these commercial providers and internal chemical lysis method was reviewed (Quick DNA Miniprep Kit). Each kit was used according to the manufacturers recommended protocol. Bead beating was standardized at 5 minutes using the MP Bio Fastprep-24, which is a high speed homogenization device. 16S rRNA genes were amplified with primers targeting v3-4 region and the amplicons were sequenced on Illumina® MiSeq™ (2×250 bp). Overlapping paired-end reads were assembled into complete amplicon sequences. The composition profile was determined based on sequence counts after mapping amplicon sequences to the known 16S rRNA genes of the eight different bacterial species (FIG. 9A-B). Significant composition bias was observed using the Human Microbiome Project Protocol (Mechanical Lysis and Heat), PowerSoil DNA Isolation Kit (Mechanical Lysis), and QIAamp DNA Stool Mini Kit (Chemical Lysis), Quick DNA Miniprep Kit (Chemical Lysis) due to inefficient lysis. The ZymoBIOMICS DNA Mini Kit which includes the 0.1 mm and 0.5 mm ultra-dense ceramic beads was the only method that enable efficient unbiased lysis.

Evaluation of Methods Using Stool:

The ZymoBIOMICS DNA Mini Kit featuring Zymo's proprietary ultra-dense ceramic BashingBeads (mixed 0.1 mm and 0.5 mm) enabled the highest lysis efficiency of gram positive bacteria in stool which is consistent with the results found using the ZymoBIOMCS Microbial Community Standard. There is a significant increase in yield and gram-positive abundance when DNA was isolated using the ZymoBIOMICS™ DNA Mini Kit. Correlated with the above results it can be concluded that unbiased DNA isolation was achieved. DNA was extracted from 200 μl of human feces suspended in PBS (10% m/v) using four different DNA extraction methods (ZymoBIOMICS™ DNA Mini Kit, Human Microbiome Project Protocol, PowerSoil DNA Isolation Kit, and QIAamp DNA Stool Mini Kit) and analyzed using 16S rRNA gene sequencing. Each kit was used according to the manufacturers recommended protocol. Bead beating was standardized to 5 minutes using the MP Bio Fastprep-24, which is a high speed homogenization device. 16S rRNA genes were amplified with primers targeting v3-4 region and the amplicons were sequenced on Illumina® MiSeq™ (2×250 bp). Overlapping paired-end reads were assembled into complete amplicon sequences. Amplicon sequences were profiled with Qiime using Greengenes 16S rRNA gene database (gg_13_8).

Lysis Efficiency Evaluated Using a Low Speed 96 Well Orbital Shaker Over a Range of Durations:

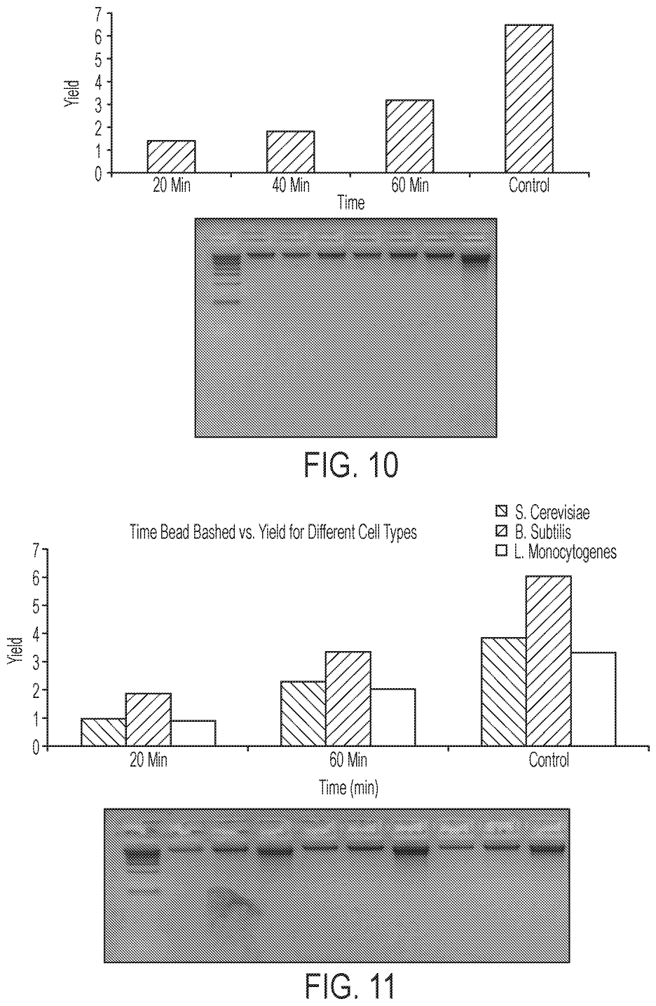

The purpose of this experiment was to determine the efficiency of a low powered 96 well shaker plate (at 2,000 rpm) for bead beating fungal and bacterial cells over increasing durations time. Pelleted Bacillus subtilis cells were resuspended in up to 200 L of DNA/RNA shield solution. To each cell/shield solution, 750 μL of Lysis Buffer was added and the entire mixture was transferred to a Zymo Bead Bashing Tube pre-filled with bashing beads. Duplicate bead beating controls were prepared which were processed on the high speed MP Bio Fastprep-24 with a 2 mL tube holder assembly, and processed at maximum speed for 5 minutes. The test samples were bead beat on an Fisher Scientific Advanced 96 Plate Vortex Shaker Plate (orbitalshaker) at 2000 rpm for 20, 40, and 60 minutes. All samples were centrifuged at 10,000×g for 1 minute prior to further processing. 400 μL of supernatant was transferred to a Zymo Spin IV Spin Filter in a Collection Tube and centrifuged at 7,000×g for 1 minute. The filtered flow through within the collection tubes was kept after centrifugation. 1,200 μL of Fungal/Bacterial DNA Binding Buffer was added to the filtrate in the Collection Tube and mixed thoroughly. 800 μL of the mixture was transferred to a Zymo Spin IIC column in a Collection Tube and centrifuged at 10,000×g for 1 minute. The flow through was discarded and the previous step was repeated. 200 μL of DNA Pre-Wash Buffer was added to the Zymo Spin IIC Column in a new Collection Tube and centrifuged at 10,000×g for 1 minute. 700 μL of Fungal/Bacterial DNA Wash Buffer was added to the Zymo Spin IIC Column in a Collection Tube at 10,000×g for 1 minute. 200 μL of Fungal/Bacterial DNA Wash Buffer was added to the Zymo Spin IIC Column in a Collection Tube at 10,000×g for 1 minute. The collection tube was emptied and a dry spin was performed at 10,000×g for 1 minute. The Zymo Spin IIC column was placed in a clean 1.5 mL microcentrifuge tube and 50 L of DNA Elution Buffer was added directly to the column matrix. After 2-3 minutes, the tube was centrifuged at 10,000×g for 1 minute to elute the DNA which was then analyzed (FIG. 10). Increasing the duration of time spent bead beating using the shaker plate increased the lysis efficiency, however even at 60 minutes using the ultra-dense ceramic mixed beads (0.1 mm and 0.5 mm) lysis was not as efficiency as using the high speed disruptor (control). The results were however, promising as this indicated that low speed disruptors were a viable option for automation with minor modifications to enhance the efficiency.

Low Speed Bead Beating of Three Tough to Lyse Organisms Using a 96-Well Orbital Plate Shaker:

The purpose of this experiment was to determine the efficiency of a low powered 96 well shaker plate (2,000 rpm) for bead beating fungal and bacterial cells at increasing durations of bead beating. Cultures of Saccharomyces cerevisiae, Listeria monocytogenes, and Bacillus subtilis were each pelleted and resuspended with 1600 μl DNA/RNA Shield. 200 μl of the suspension was used for each preparation. To each cell/shield solution, 750 μL of Lysis Buffer was added and the entire mixture was transferred to a Zymo Bead Bashing Tube pre-filled with bashing beads. Duplicate bead beating controls were prepared which were processed on the high speed MP Bio Fastprep-24 with a 2 mL tube holder assembly, and processed at maximum speed for 5 minutes. The test samples were bead beat using the Fisher Scientific Advanced 96 Plate Vortex Shaker Plate and bead beat at 2000 rpm for 20 and 60 minutes. All samples were centrifuged at 10,000×g for 1 minute prior to further processing. 400 μL of supernatant was transferred to a Zymo Spin IV Spin Filter in a Collection Tube and centrifuged at 7,000×g for 1 minute. The filtered flow through within the collection tubes was kept after centrifugation. 1,200 μL of Fungal/Bacterial DNA Binding Buffer was added to the filtrate in the Collection Tube and mixed thoroughly. 800 μL of the mixture was transferred to a Zymo Spin IIC column in a Collection Tube and centrifuged at 10,000×g for 1 minute. The flow through was discarded and the previous step was repeated. 200 μL of DNA Pre-Wash Buffer was added to the Zymo Spin IIC Column in a new Collection Tube and centrifuged at 10,000×g for 1 minute. 700 μL of Fungal/Bacterial DNA Wash Buffer was added to the Zymo Spin IIC Column in a Collection Tube at 10,000×g for 1 minute. 200 μL of Fungal/Bacterial DNA Wash Buffer was added to the Zymo Spin IIC Column in a Collection Tube at 10,000×g for 1 minute. The collection tube was emptied and a dry spin was performed at 10,000×g for 1 minute. The Zymo Spin IIC column was placed in a clean 1.5 mL microcentrifuge tube and 50 μL of DNA Elution Buffer was added directly to the column matrix. After 2-3 minutes, the tube was centrifuged at 10,000×g for 1 minute to elute the DNA which was then analyzed. The DNA was quantified using Nanodrop and electrophoresed on a 1% agarose gel (FIG. 11). Increasing the amount of time spent bead beating using the shaker plate increased the lysis efficiency, however as expected even after 60 minutes it did not achieve the efficiency of the control (high speed disruptor). Note that the relative yields remain substantially similar within each set indicating that bias is being mitigated due to the fact that the lysis is reasonably uniform between these three tough to lyse organisms This further exemplifies proof of principle that this method can be applied to automation (including high throughput) in an inexpensive simple format.

Bead Beating Lysis Efficiency Test Hamilton Heater Shaker Module:

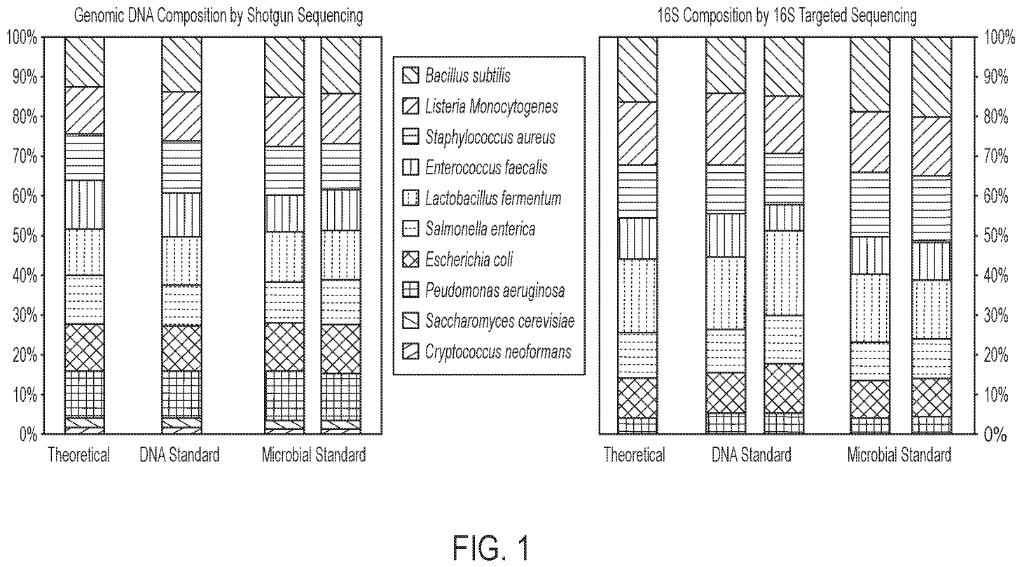

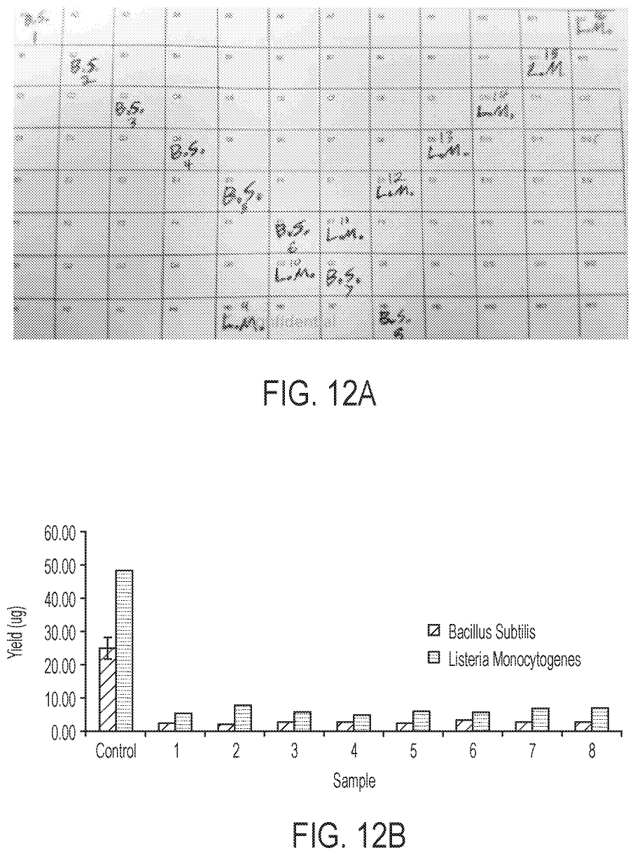

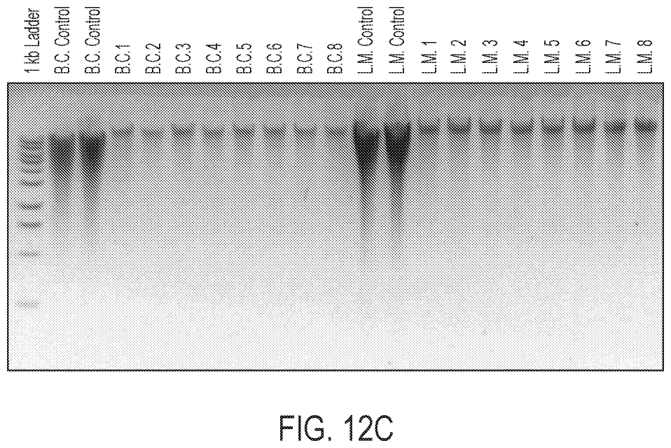

To test the lysis efficiency of L. monocytogenes and B. subtilis, mechanical homogenization was performed by bead beating on an on-deck vortex with the Hamilton ML_STAR system. Using 200 μL of each of the cultured organisms, bead beating was performed at different locations (FIG. 1A) throughout a 96-well deep well block sealed with cover foil on the on-deck Hamilton Heat Shaker unit for 10 minutes. Controls were performed by bead beating in the MP-Biomedicals Fastprep-24 sample homogenizer and the ZR BashingBead Lysis Tubes. Each sample was then removed from the beads and purified using the ZymoBIOMICS DNA Miniprep kit protocol. Samples were quantified using Nanodrop spectrophometry and agarose gel electrophoresis (FIGS. 12A-12C). This experiment determined that bead beating for 10 minutes on the Hamilton ML_STAR system produced inefficient lysis and did not fully lyse either of the model organisms.

Bead Beating Lysis Efficiency Time Trial Using the Hamilton Heater Shaker Module:

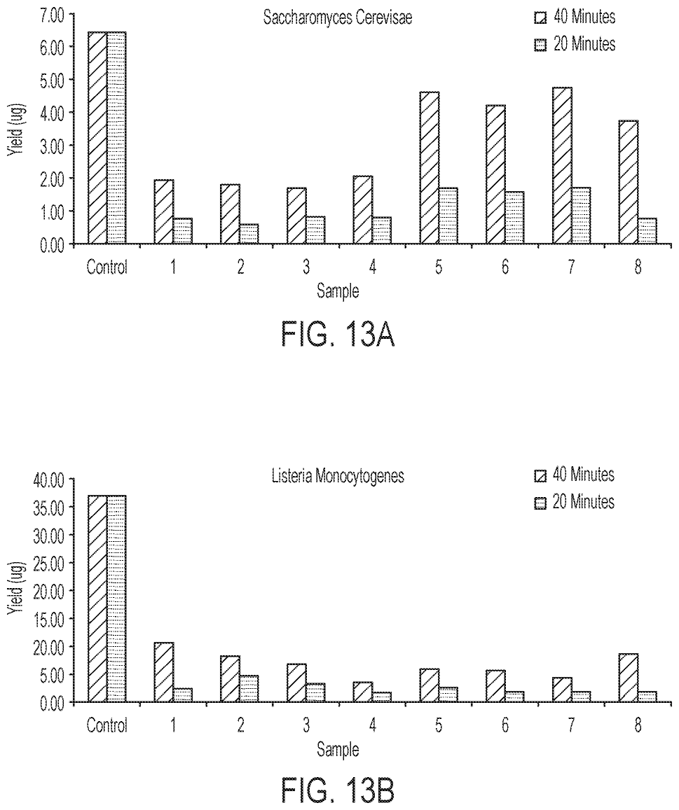

Thus, a time trial was evaluated to determine the length of time needed for more efficient lysis. To test the lysis efficiency of Listeria monocytogenes and Saccharomyces cerevisiae at different time points when bead beating was performed on a Hamilton ML_STAR Heater Shaker module. Cultures of Saccharomyces cerevisiae and Listeria monocytogenes were each pelleted and resuspended with DNA/RNA Shield. Using 200 μl of each of the cultured organism suspensions, bead beating was performed at different locations throughout a 96-well Deep Well Block sealed with cover foil on the on-deck Hamilton Heater Shaker unit for 20 and 40 minutes. Controls were performed by bead beating in the MP-Biomedicals Fastprep-24 sample homogenizer and the ZR BashingBead Lysis Tubes containing 0.1 mm and 0.5 mm ultra-dense ceramic beads. Each sample was then removed from the beads and purified using the ZymoBIOMICS DNA Miniprep kit protocol (D4300). Samples were quantified using Nanodrop spectrophotometry and agarose gel electrophoresis (FIG. 13). This experiment determined that in order to achieve improved lysis efficiency the sample must be bead beat for at least 40 minutes on the Hamilton ML_STAR system. It was discovered that the lysis efficiency was greatly improved at the edges of the plate vs. the center, indicating that the amplitude of the shakers orbit plays a role in the lytic efficiency at each position. This experiment also indicated that larger yeast organisms (Saccharomyces cerevisiae) can be lysed in addition to bacteria tested previously using this on-deck orbital shaker.

Evaluation of Bead Beating Bias Using the Hamilton Heater Shaker Module:

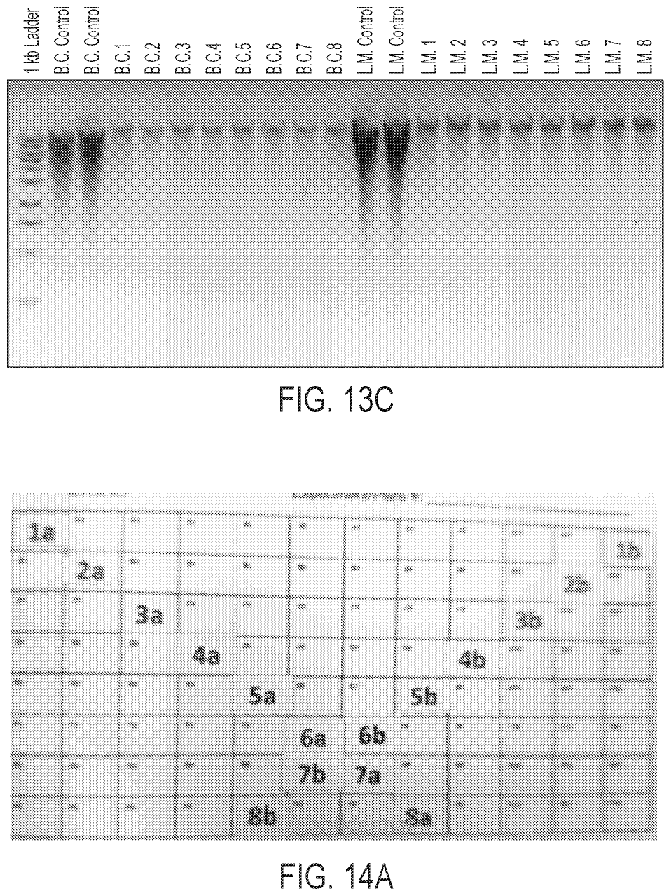

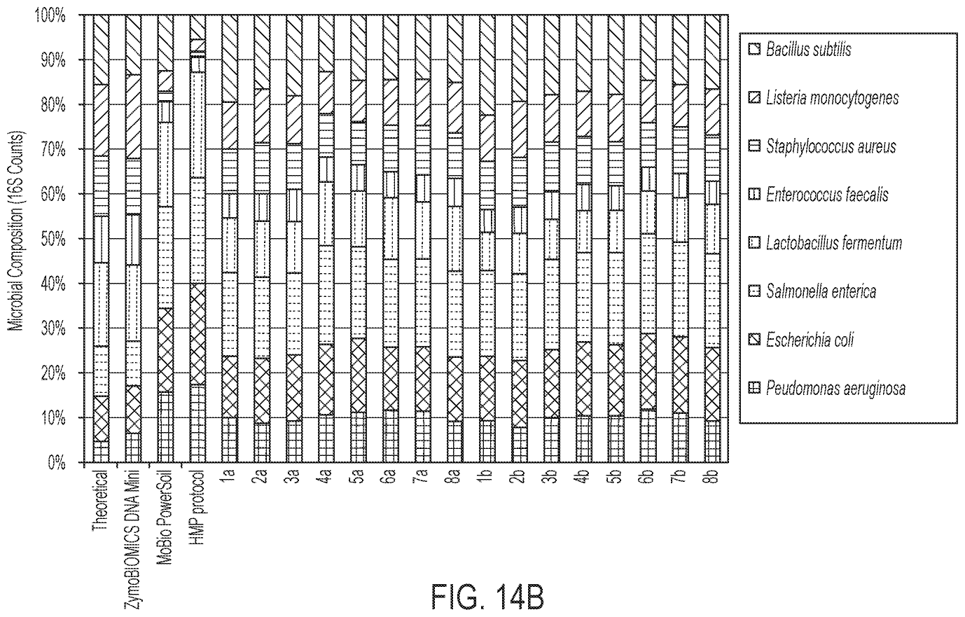

In order to evaluate the lytic bias present when using the Hamilton Heater Shaker system for bead beating a mock microbial community was utilized. 100 μl of ZymoBIOMICS Microbial Community Standards was processed and bead beating was performed at different locations (FIG. 13A) throughout a 96-well Deep Well Block sealed with cover foil on the on-deck Hamilton Heater Shaker unit for 40 minutes. Controls were performed by bead beating in the MP-Biomedicals Fastprep-24 sample homogenizer and the ZR BashingBead Lysis Tubes (Microbe). Each sample was then removed from the beads and purified using the ZymoBIOMICS DNA Miniprep kit protocol (D4300). Each sample was then analyzed using 16S rRNA gene sequencing to determine the microbial composition present in each preparation (FIG. 14B). Bead beating on the Hamilton ML_STAR system lead tobias compared to the theoretical composition of the microbial standard and the results of the optimized ZymoBIOMICS DNA Mini Kit which uses the (0.1 mm and 0.5 mm ultra-dense ceramic beads) on the MP-Biomedical Fastprep-24 controls. The bias was increased near the center of the plate and was decreased towards the edges of the plate, agreeing with the results of the previous experiment. However, despite the presence of some bias towards gram negative organisms, it was substantially reduced as compared to the Mo Bio PowerSoil Kit and the Human Microbiome Project Protocol which saw respectively a 56% and 76% deviation from the actual abundance of gram positive organisms. Adaptation of a non-optimized low speed orbital shaker plate with Zymo's proprietary ultra-dense 0.1 mm and 0.5 mm ceramic BashingBeads yielded improved accuracy and substantially improved accuracy as compared to the most cited methods utilizing a high speed disruptor (MP-Biomedical Fastprep-2). With minor improvements to the motion of orbital shaker the system could rapidly be inexpensively adapted to a fully automated high throughput unbiased microbial nucleic acid purification system. The improvements could be used to optimize the system would be clear to an expert in the field, however some examples that have been contemplated include the speed (number of oscillations/second) and the direction of motion.

Bead Bashing in Binding Solution (Genomic Lysis Buffer):

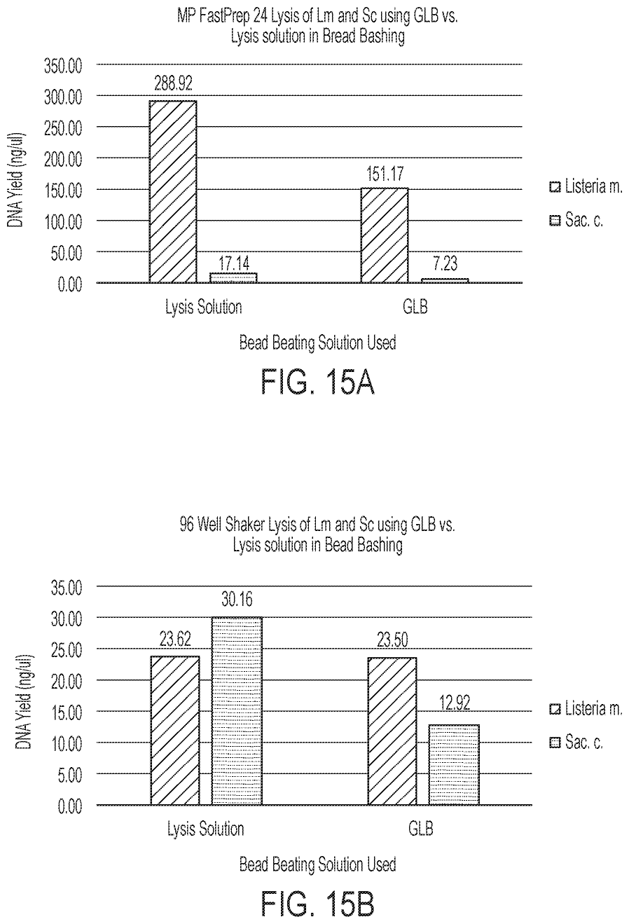

This experiment evaluated beating solutions that could simultaneously serve as a binding agent. This is particularly advantageous for bead mover technologies, such as that used by the Promega Maxwell 16, as the addition of liquid post bead beating would require additional liquid handling mechanics such as a pipette or peristaltic dispenser. Zymo Research's Genomic Lysis Buffer which contains high molarity guanidine thiocyanate (serves to facilitate binding) and Zymo Research's Lysis Solution which is a high concentration EDTA solution (does not serve to facilitate binding) were both evaluated. Pure cultures of Listeria monocytogenes and Saccharomyces cerevisiae were pelleted and resupended in water. Suspensions for each sample were bead baeat in the Genomic Lysis Buffer or Lysis and solution further purified using the Quick DNA Miniprep Kit. Bead bashing was performed using the 2 ml Lysis Tube individual format on the MP FastPrep 24 at 6.5 m/s for 5 minutes. The 96-well format was tested on the Fisher Scientific Advanced 96 Shaker plate, 200 RPM for 30 minutes. Each sample was then removed from the beads and purified using the ZymoBIOMICS DNA Miniprep kit protocol (D4300). Each sample was then quantified using Nanodrop (FIGS. 15A-15B). High speed shakers appeared to induce a reduction in yields overall, while low speed shakers appeared to have little or no effect on the lysis of Listeria monocytogenes but lead to reduced yields for Saccharomyces cerevisiae.

Time Course Using GLB as Bead Bashing Solution Compared with Lysis Solution:

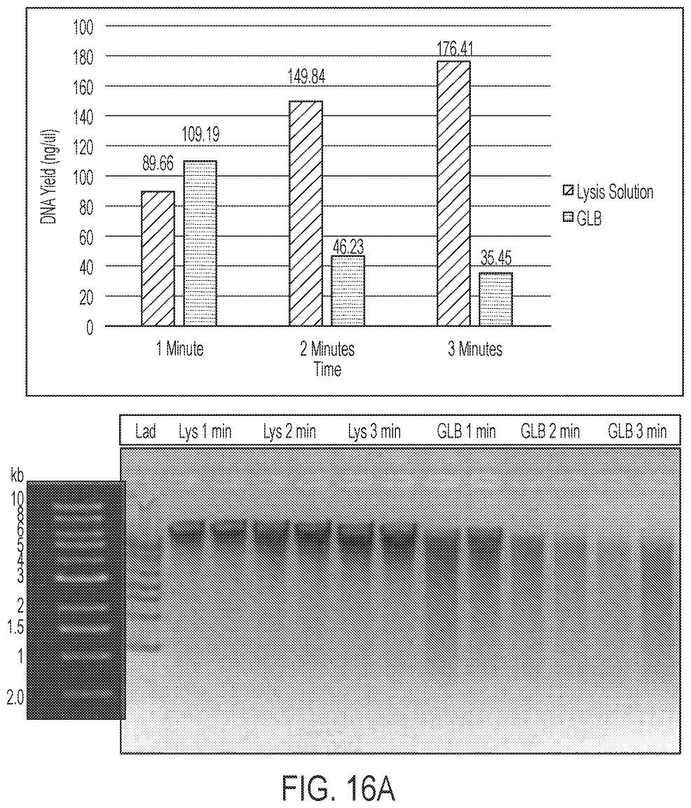

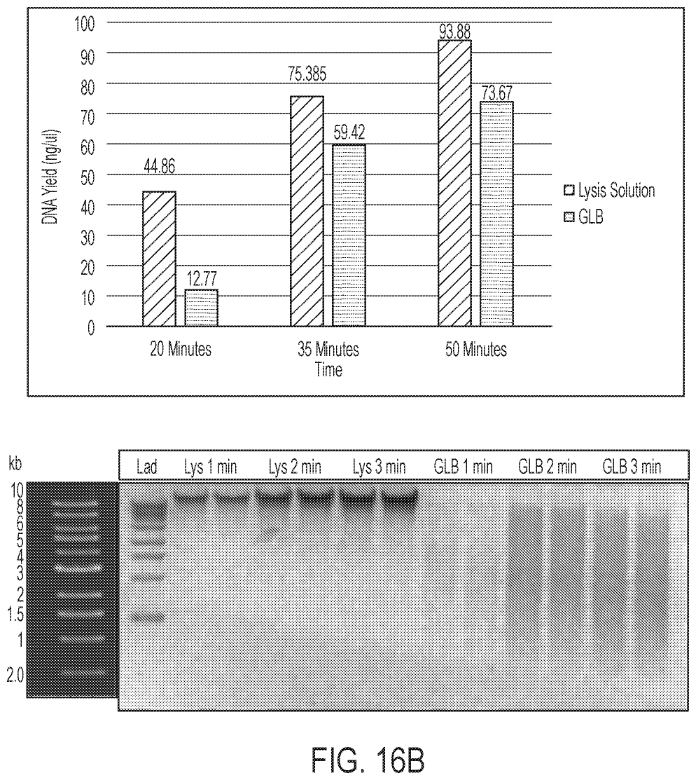

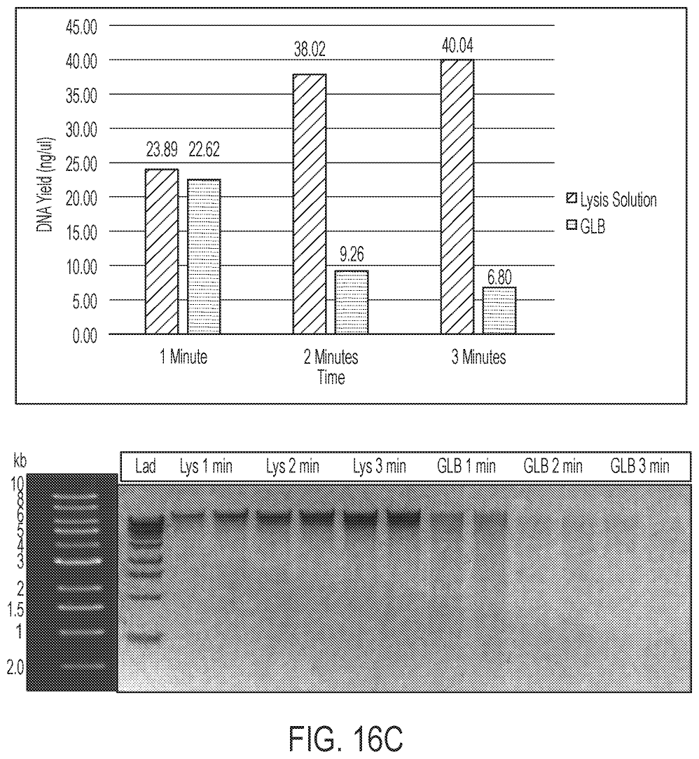

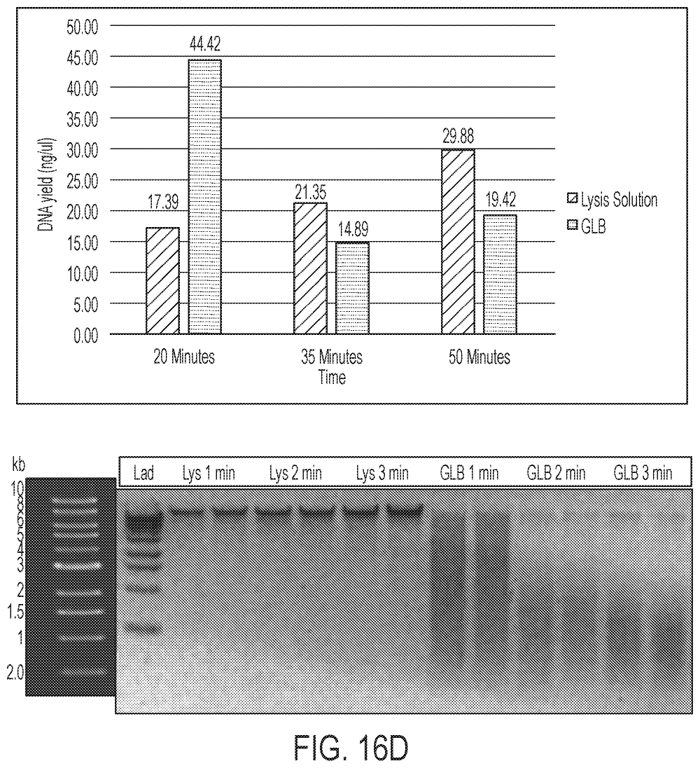

Listeria m. and Saccharomyces c. (2 difficult to lyse organisms, one small and one large) were used to test GLB in bead bashing. 50 ul of Listeria m. and 500 ul of Saccharomyces c. pure culture were used and bead beating was performed in GLB or Lysis solution. Bead bashing was performed using the 2 ml Lysis Tube individual format on the MP FastPrep 24 at 6.5 m/s for 1, 2, and 3 minutes. The 96-well format was tested on the Fisher Scientific Advanced 96 Shaker plate, 200 RPM for 20, 35, and 50 minutes. 3 different time points were ised for each to see when GLB as a bead bashing solution seems to take effect. Each sample was removed from beads and then extracted using the ZymoBIOMICS DNA Mini Kit (D4300). Each sample was then subjected to Nanodrop analysis and 1% agarose gel electrophoresis (FIGS. 16A-16D). It was observed that GLB seems to reduce overall yield for all groups.

Evaluation of Automated On-Deck Bead Beating Using the Hamilton ML_STAR System:

In order to illustrate a fully automated reduced bias purification system the Hamilton ML_STAR system was used for both bead beating and purification. Bead beating was performed using 20 mg of fecal sample. 200 μl of 10% stool in DNA/RNA Shield Solution was processed by bead beating at different locations (Table 7) throughout a 96-well Deep Well Block sealed with cover foil on the on-deck Hamilton Heater Shaker unit for 40 minutes. Controls were performed by bead beating in the MP-Biomedicals Fastprep-24 sample homogenizer and the ZR BashingBead Lysis Tubes (Microbe). Each sample was then removed from the beads and purified using the ZymoBIOMICS 96 Magbead DNA kit protocol (D4302). Each sample was then quantified using spectrophotometric analysis to determine the concentration and purity of DNA present. As previously observed using the mock microbial community standards bead beating using the non-optimized Hamilton orbital shaker unit led to reduced yields compared to the results compared to the control that used a high-speed disruptor. The yields were decreased near the center of the plate and was increased towards the edges of the plate, agreeing with the results of previous experiments indicating that the rotational orbit creates bias in the lysis efficiency of the sample. This proof principle taken with the mock microbial community data indicates that a fully automated system could immediately be utilized by the community at substantially reduced cost and substantially reduced bias. With minor improvements to the motion of orbital shaker the system could rapidly be inexpensively adapted to a fully automated high throughput unbiased microbial nucleic acid purification system. The improvements could be used to optimize the system would be clear to an expert in the field, however some examples that have been contemplated include the speed (number of oscillations/second) and the direction of motion. Based on this proof of concept other devices described in the disclosure could be contemplated and readily assembled.

TABLE 6
Analysis of DNA

| Sample | Concentration (ng/μL) | A260/280 | A260/230 | Yield (μg) |
|---|---|---|---|---|
| 1 | 57.32 | 2 | 1.78 | 2.87 |
| 2 | 57.52 | 2.01 | 2.13 | 2.88 |
| 3 | 49.53 | 2.01 | 2.2 | 2.48 |
| 4 | 38.26 | 1.99 | 2.15 | 1.91 |
| 5 | 28.83 | 2.03 | 2.11 | 1.44 |
| 6 | 57.53 | 2.03 | 1.85 | 2.88 |
| 7 | 57.2 | 2.06 | 2.08 | 2.86 |
| 8 | 55.91 | 2.08 | 2.21 | 2.80 |
| 9 | 57.74 | 1.98 | 2.05 | 2.89 |
| 10 | 57.5 | 1.99 | 1.93 | 2.88 |
| Control | 173.23 | 1.93 | 2.06 | 8.66 |
| Control | 178.56 | 1.98 | 2 | 8.93 |

TABLE 7
Different locations throughout a 96-well Deen Well Block

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | | | | | | | | |
|---|---|---|---|---|---|---|---|---|---|---|
| A | | | | | | | | | | |
| B | 1 | | | | | | | | | |
| C | | 2 | | | | | | | | |
| D | | | 3 | | | | | | | |
| E | | | | 4 | | | | | | |
| F | | | | | 5 | | | | | |
| G | | | | | | 6 | | | | |
| H | | | | | | | 7 | 8 | 9 | 10 |

### Example 2—Further Characterization of Lysis Method

To further characterize and optimize the automated lysis method of Example 1, the microbial sample was loaded into the wells of a plate with a rectangular bashing magnet in each well. The driving magnet was placed under the plate and used to apply varying offsets of oscillation such as 8 mm, 15 mm, and 20 mm. FIGS. 17A and 17B show plots of averaged sample percentage yield for mechanical lysis of Listeria and Saccharomyces cerevisiae cells. The samples were tested with stationary, 8 mm, or 15 mm offset runs. Each chart shows a point plot (solid line) and a corresponding linear trending plot (dotted line) for each of the tests. The 15 mm test showed qualitative improvements in percentage yield for mechanical lysis across all test positions in the test matrix, where B1/D1 are peripheral, A2/E2 are intermediate, and B4/D4 are central test positions of the test matrix.

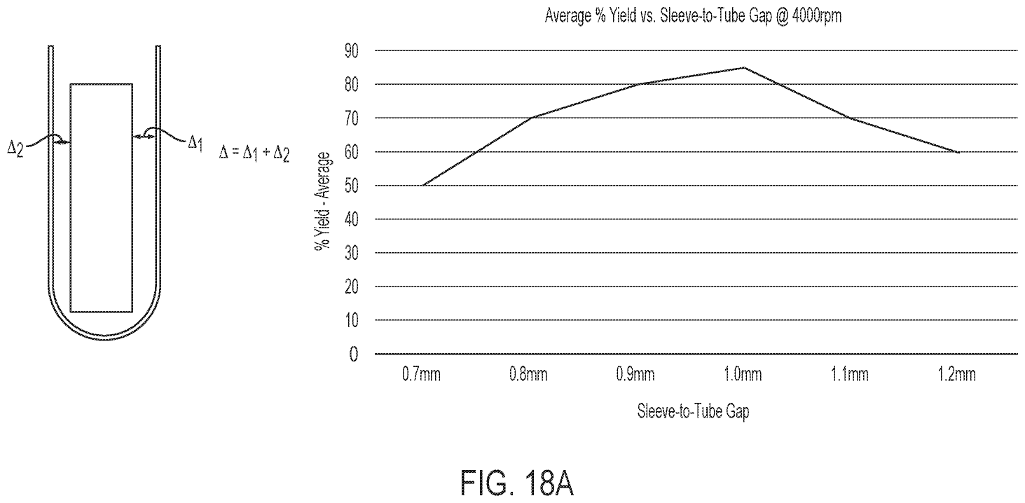

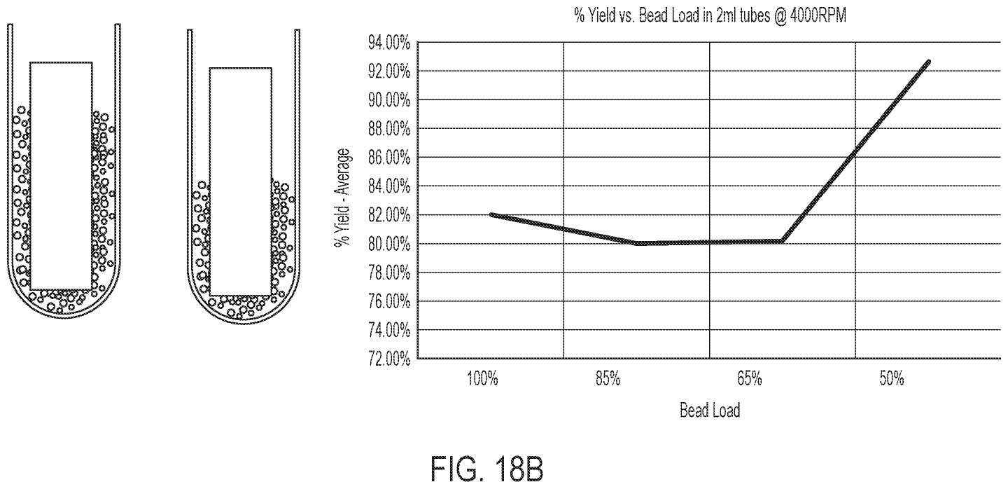

Next, the average percent yield was determined based on the gap between the sleeve and the tube. A distance of 0.7 mm up to 1.2 mm between the sleeve and the tube was tested at 4000 rpm. It was found that around 1 mm distance resulted in the highest percent yield (FIG. 18A).

Another parameter that was tested for the lysis method was the bead load in the tubes. A bead load of 50%, 65%, 85%, or 100% was tested for percent yield at 4000 rpm. It was found that the lower bead load of 50% resulted in the higher percent yield of nucleic acids (FIG. 18B).

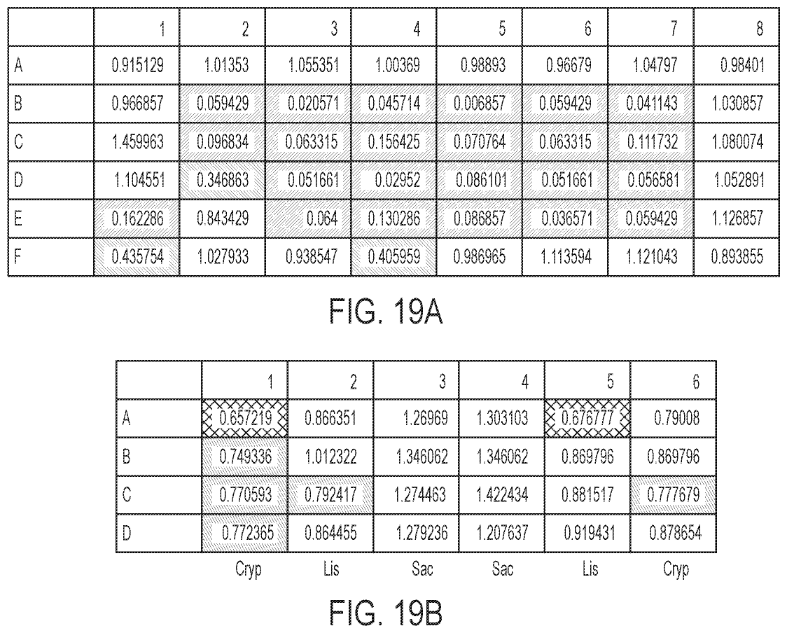

The pulling force ratio of the drive magnet to the bashing magnet was tested next as well as the effect of the distance between the bashing magnets. The 48-well plate was used for a spacing test using the mixed microbial sample, and it was found that high-yield bashing is achieved with the perimeter wells due to the lower magnetic coupling at the middle wells (FIG. 19A). The spacing test using the 24-well plate showed high-yield bashing for Listeria and Saccharomyces (FIG. 19B).

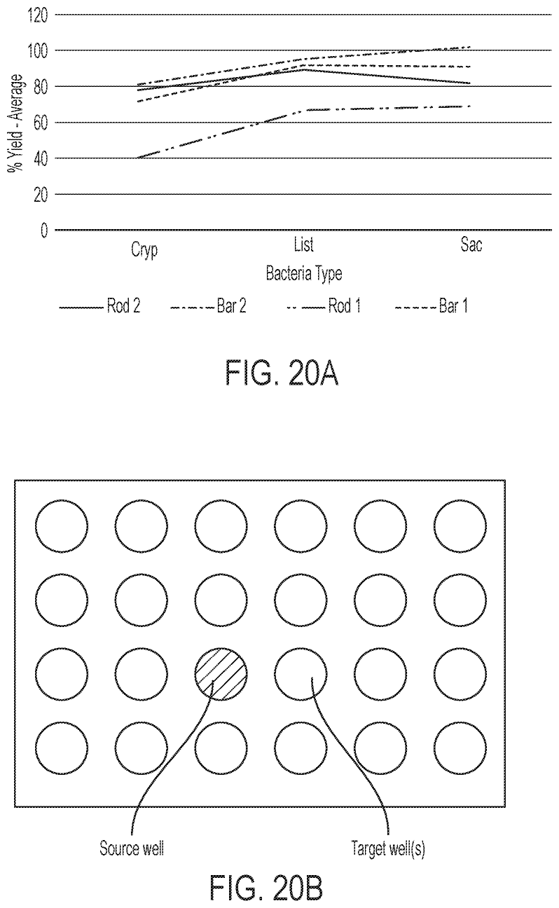

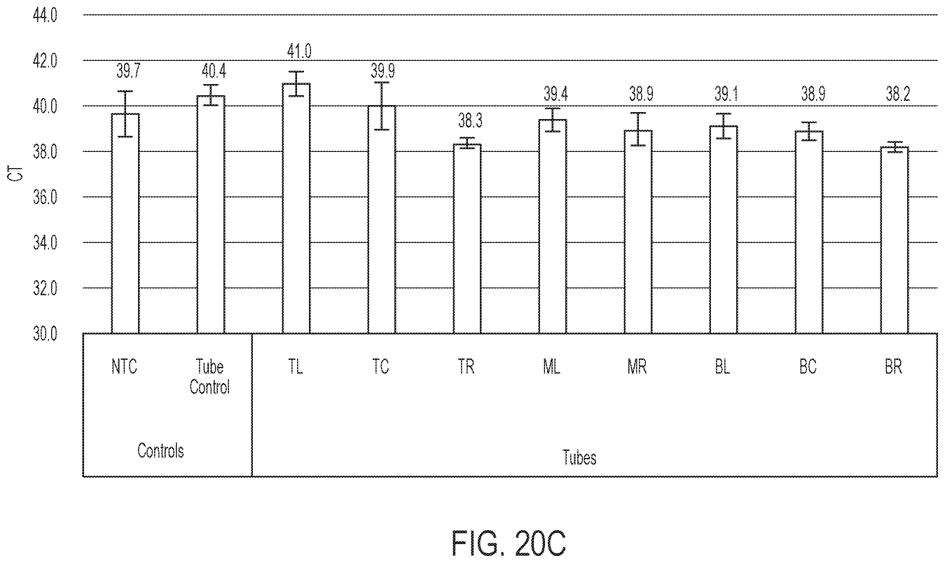

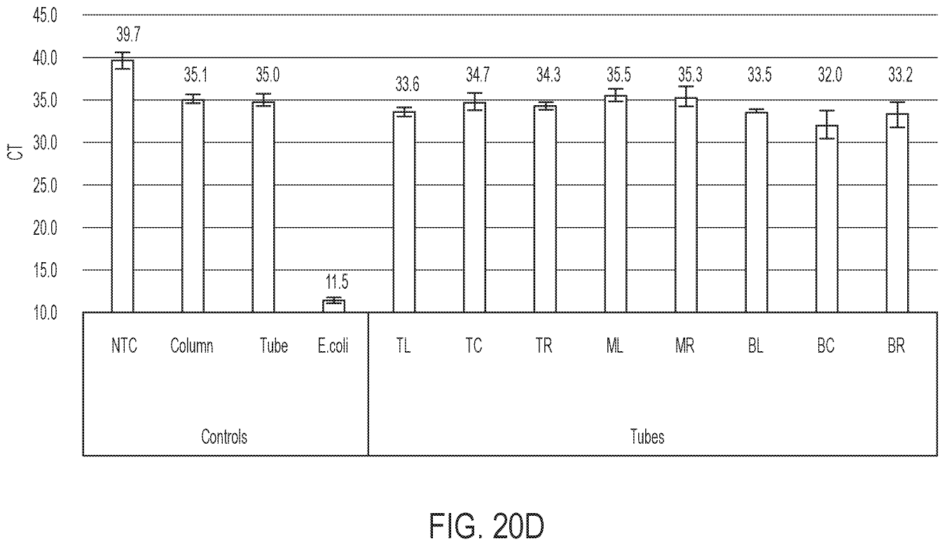

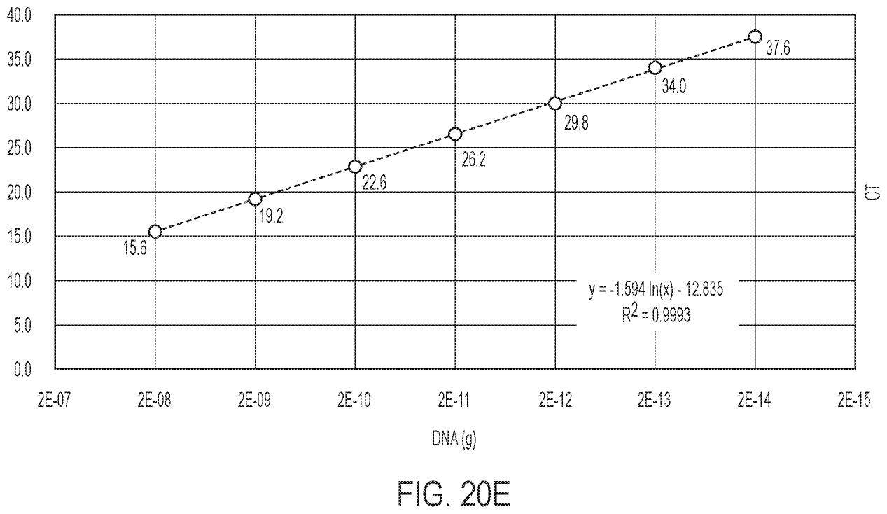

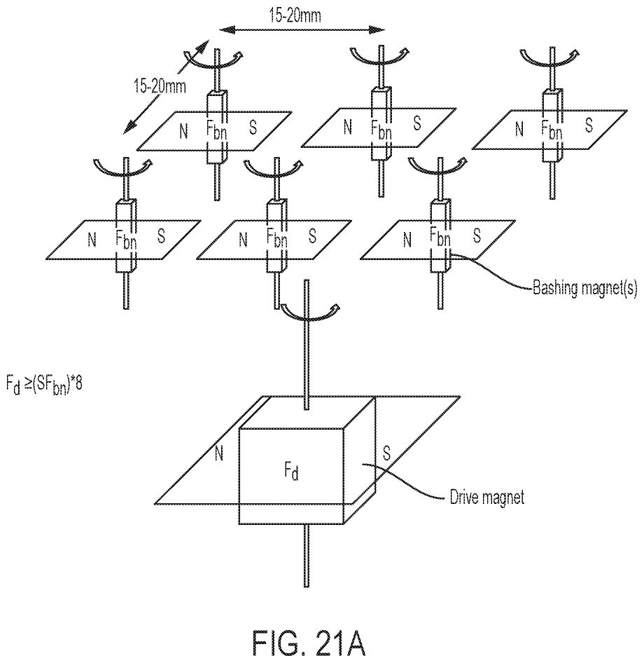

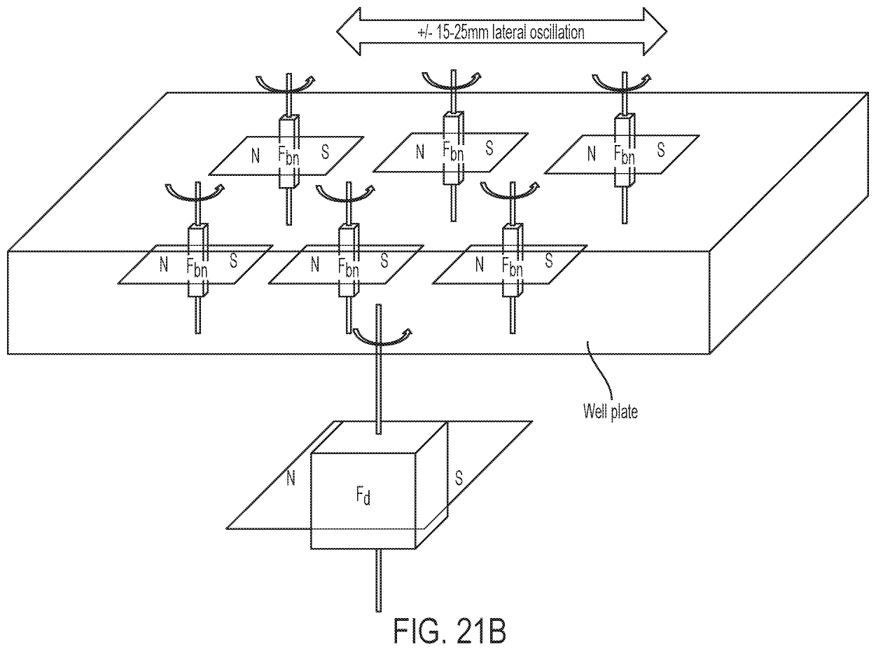

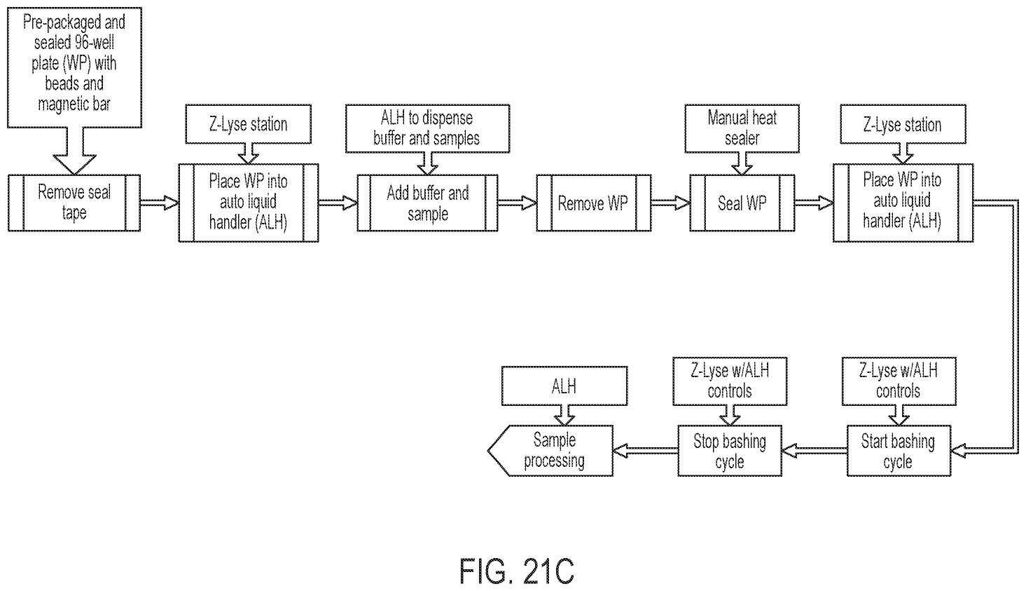

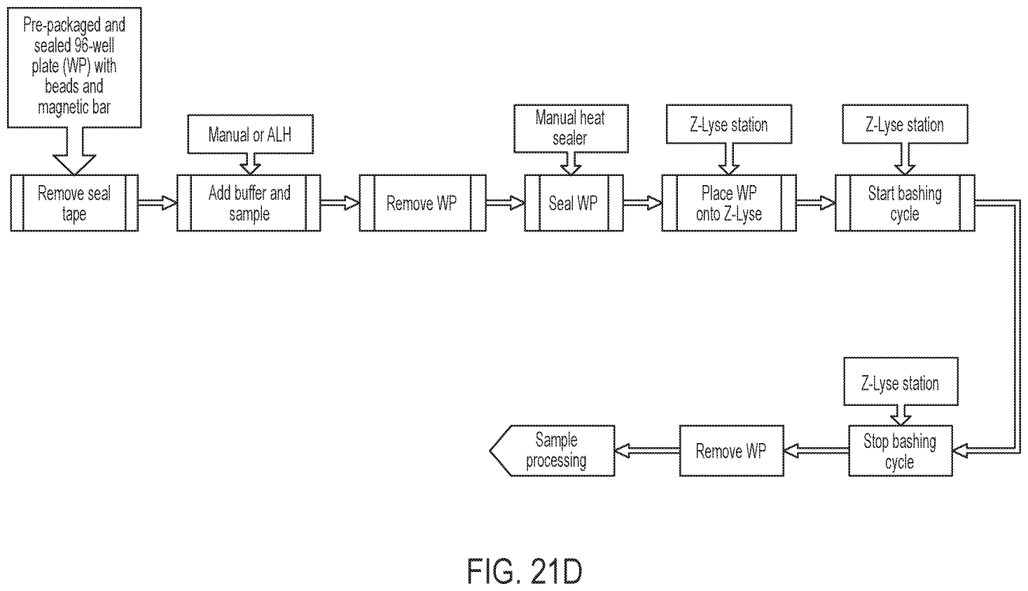

The effect of the shape of the bashing magnet on percent yield was also tested. It was found that the rectangular bar resulted in a higher yield than the circular rod (FIG. 20A). The tests were performed with 14 mm×74 mm tubes in a 24-well pattern (FIG. 20B). The source well was filled with the lysis solution, bashing magnet, beads and the microbial sample. The target wells were filled with pure water. The tests showed no visible splash outside of the source tube and no cross-contamination between the source and the target tubes.

Thus, it was found that high-yield lysis in the range of 80% to 100% was achieved in a 24-well format (i.e., 4×6) within the standard well plate footprint for tough-to-lyse microbes such as Saccharomyces and Listeria. In addition, the percent yield was found to be improved by using a lateral offset between 15-25 mm during the bashing cycle.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Ariefdjohan et al., Nutr. J., 2010.
Beckers et al., 14th International Conference on Miniaturized Systems for Chemistry and Life
Sciences, 2010.
Bimboim and Dolly, 1979.
Bimboim, 1983.
Chomczynski & Sacchi, 1987.
Chomczynski et al., 1989.
Clewell and Helinski, 1970.
European Patent No. 0,462,257
European Patent No. 0,679,430
European Patent No. 1,393,797
Grassman & Deffner, 1953.
Holmes and Quigley, 1981.
International Patent Publication No. WO 98/32838
International Patent Publication No. WO1996012959
Japanese Patent No. 10-277434
Japanese Application No. 2007-237036
Kennedy et al., PLOS One, e88982, 2014.
Kirby, 1956.
Lis and Schleif, 1975.
Marko et al., 1981.
Price et. al., 2009
Purcell et al., Am. J. Phys, 1977.
Rashmi et al., Genome Biol Genome Biology 16.1, 2015.
Rogers and Bendich, 1985.
Ruggiere et al. Springer Protocols Handbooks, Sample Preparation Techniques for Soil, Plant,
and Animal Samples, 41-52, 2016
Sambrook et al., 2001.
Sinha et al., 2015.
Stulberg et al., Nat. Microbiol Nature Microbiology 1.1, 2016.
Tan & Yiap, 2009.
U.S. Pat. No. 4,673,297
U.S. Pat. No. 4,990,130
U.S. Pat. No. 4,990,130
U.S. Pat. No. 5,075,430
U.S. Pat. No. 5,702,950
U.S. Pat. No. 5,705,628
U.S. Pat. No. 5,898,071
U.S. Pat. No. 6,027,945
U.S. Pat. No. 6,190,032
U.S. Pat. No. 6,284,470
U.S. Pat. No. 6,673,631
U.S. Pat. No. 6,942,806
U.S. Pat. No. 7,078,224
U.S. Pat. No. 7,495,090
U.S. Pat. No. 7,622,046
U.S. Pat. No. 8,430,247
U.S. Pat. No. 9,051,563
U.S. Patent Publication No. 2009/086573
U.S. Patent Publication No. 2010/218620
U.S. Patent Publication No. 2011/286298
U.S. Patent Publication No. 2015/0036450
Vogelstein et al., 1979.
Zoetendal et al., Syst Appl Microbiol., 24(3):405-10, 2001.

1. An automated method for reduced-biased nucleic acid isolation from a plurality of samples comprising:
(a) disposing the plurality of samples into an array of sample containers; and
(b) applying a mechanical force to the sample containers, wherein each sample container comprises the sample and a bead or a plurality of beads for disrupting microbial cells and viruses, thereby providing a unbiased release of microbial nucleic acids.

2. The method of claim 1, wherein the plurality of beads are loaded in the sample container at 40-60% by volume.

3. The method of claim 1, wherein the plurality of beads comprise beads of different materials, different sizes, different shapes or a combination thereof.

4. The method of claim 3, wherein the beads are substantially spherical and comprise an average diameter of between 0.01 and 1.0 mm.

5. (canceled)

6. The method of claim 1, wherein the bead is substantially spherical.

7. (canceled)

8. The method of claim 1, wherein the bead is composed of a ceramic.

9. The method of claim 1, wherein the sample containers are in a 24-well, 48-well, or 96-well format.

10. (canceled)

11. The method of claim 1, wherein the plurality of samples comprise viruses, bacterial cells, fungal cells, algal cells, plant cells, animal cells, archaeal cells, protozoans or a mixture thereof.

12-15. (canceled)

16. The method of claim 1, wherein applying a mechanical force comprises subjecting the sample container to oscillation.

17. The method of claim 16, wherein the oscillation is further defined as lateral oscillation, horizontal oscillation, vertical oscillation, orbital oscillation or a mixture thereof.

18. The method of claim 1, wherein the oscillation is further defined as orbital oscillation.

19. The method of claim 16, wherein the sample container further comprises a bashing magnet.

20. The method of claim 19, wherein the bashing magnet is rectangular or cylindrical.

21. The method of claim 19, wherein the bashing magnet is a rectangular bar.

22. The method of claim 19, wherein the bashing magnet and edge of the sample container comprise a gap of 0.7 to 1.2 mm.

23. The method of claim 19, wherein the bashing magnet and edge of the sample container comprise a gap of 0.9 to 1.1 mm.

24. The method of claim 20, wherein subjecting the sample container to oscillation comprises using a drive magnet to move the bashing magnet.

25-27. (canceled)

28. The method of claim 24, wherein the drive magnet to bashing magnet pulling force ratio is from 8:1 to 10:1.

29-42. (canceled)

43. A kit comprising (1) a plurality of high density beads; (2) a device for applying an automated mechanical force to a sample container; and (3) a control sample.

44-47. (canceled)

48. A device for the purification of nucleic acids comprising an automated system comprising a plurality of sample containers, each container comprising cell lysis beads and a system for providing mechanical force to the containers, wherein the device provides lysis of microbial samples with a reduced bias.

49-54. (canceled)
